(19) United States
(12) Patent Application Publication
(10) Pub. No.: US 2021/0212581 A1
(43) Pub. Date: Jul. 15, 2021

(54) METHOD AND APPARATUS FOR PROVIDING BIOMETRIC INFORMATION BY ELECTRONIC DEVICE

(71) Applicant: Samsung Electronics Co., Ltd., Gyeonggi-do (KR)

(72) Inventors: Eunsun KIM, Gyeonggi-do (KR); Hyogil KIM, Gyeonggi-do (KR)

(21) Appl. No.: 17/059,647

(22) PCT Filed: Jun. 13, 2019
(86) PCT No.: PCT/KR2019/007142

§ 371 Date: Nov. 30, 2020

(30) Foreign Application Priority Data
Jun. 14, 2018 (KR) 10-2018-0067987

(51) Int. Cl. A61B 5/024; A61B 5/145; A61B 5/00

(52) U.S. Cl. CPC A61B 5/02405; A61B 5/7435; A61B 5/7282; A61B 5/14532

(57) ABSTRACT

Various embodiments of the present invention provide a method and apparatus for measuring a users' biometric information and providing information related to the biometric information by an electronic device. An electronic device according to various embodiments of the present invention may comprise a sensor module, a display device, and a processor, wherein the processor: acquires, on the basis of the sensor module, biometric information of a user and place information related to the user; matches the biometric information with the place information; displays an interface including biometric information for a predetermined period of time through the display device; determines a place of a region selected by the user and a duration corresponding to the place in the interface; and specifies the duration and highlightedly displays biometric information within the duration in the interface. Various embodiments are possible.

### TECHNICAL FIELD

Various embodiments provide a method and apparatus for acquiring (or measuring) biometric data of a user, and providing biometric information related to the user by using the acquired biometric data, by an electronic device.

### BACKGROUND ART

With the development of digital technology, various types of electronic devices such as a mobile communication terminal, a smartphone, a tablet personal computer (PC), a laptop computer (e.g., a notebook), a personal digital assistant (PDA), a wearable device, or a digital camera, etc., have been widely used.

Recently, various services (or functions) are provided for user's health care, using an electronic device. For example, the electronic device may acquire biometric data related to the user's health care, and may provide various types of health information (e.g., heart rate information, stress information, etc.) to the user, or may provide exercise coaching according to the biometric data to the user, based on the acquired biometric data.

Generally, biometric information measurement by the electronic device may be performed based on the user's desire, or may be performed regardless of the user's desire. For example, the user may execute an application (e.g., a health care application) allowing biometric data measurement in the electronic device, may prepare measurement of the user's biometric data (e.g., may prepare a sensor related to biometric data to be measured, for biometric recognition), and may perform an operation such as maintaining a fixed posture so that consecutive measurement can be performed for a configured measurement time in order to acquire the corresponding biometric data. In another example, the electronic device may be worn on a part (e.g., wrist, etc.) of the user's body, and may measure biometric data based at least in part on constant and periodical measurement, measurement upon the user's request, or detection of a configured interruption, while the electronic device is worn on the user's body.

### DISCLOSURE OF INVENTION

### Technical Problem

The electronic device may provide various types of biometric-data-based health information (e.g., heart rate information and stress information) to the user, the biometric data being measured as described above. For example, the electronic device may display a visual interface (e.g., a user interface (UI)) for providing the user's health information (e.g., heart rate information) generated based on the biometric data, through a display. However, the health information provided by the electronic device may simply include the concept of time only. For example, the electronic device provides, based on a temporal factor only, simple biometric information, such as provision of related biometric information by using biometric information measured at the time point at which the user desires health information, or biometric data measured immediately before the user desires health information, or provision of pieces of accumulated information related to biometric information which has been provided to the user until now.

Various embodiments provide a method and apparatus for providing biometric information in association with a spatial place (e.g., home, an office, a car, a performance venue, etc.) of a user.

Various embodiments provide a method and apparatus for acquiring biometric information at least in association with a place related to a user, and providing, based on the biometric information, coaching related to a user context at the corresponding place.

Various embodiments provide a method and apparatus for classifying biometric data according to a place, specifying, as a visual effect, biometric information related to biometric data of a place corresponding to a user's input (e.g., touch), and providing the same to the user.

Various embodiments provide a method and apparatus for analyzing, based on various types of context information (e.g., a time, a place, a device usage log, etc.), biometric data collected by multiple electronic devices, storing related insight information, and recognizing a user context to provide coaching to the user through the insight information appropriate for corresponding context.

### Solution to Problem

An electronic device according to various embodiments of the disclosure may include: a sensor module, a display device, and a processor, wherein the processor: acquires, using the sensor module, biometric information of a user and place information related to the user; matches the biometric information with the place information; displays an interface including biometric information for a predetermined period of time through the display device; determines a place of a region selected by the user and a duration corresponding to the place in the interface; and specifies the duration and displays biometric information by highlighting the biometric information within the duration in the interface.

An operation method of an electronic device according to various embodiments of the disclosure may include: acquiring, using a sensor module, biometric information of a user and place information related to the user; matching the biometric information with the place information; displaying an interface including biometric information for a predetermined period of time through the display device; determining a place of a region selected by the user and a duration corresponding to the place in the interface; and specifying the duration and displaying biometric information by highlighting the biometric information within the duration in the interface.

In order to solve the above problems, various embodiments of the disclosure may include a computer-readable recording medium in which a program for executing the method by a processor is stored.

### Advantageous Effects of Invention

According to an electronic device and an operation method thereof according to various embodiments, the electronic device may provide biometric information and coaching based on the biometric information, in association with a user's spatial place (e.g., home, an office, a car, a performance venue, etc.), instead of a geographical (or regional) location. According to various embodiments, the electronic device may acquire biometric information in association with a place related to the user, thereby providing an effect of preventive health care through coaching related to a user context at the corresponding place, based on the biometric information.

According to various embodiments, the electronic device may classify biometric data according a duration for each place, and may specify, as a visual effect, biometric information related to biometric data of a place corresponding to a user input (e.g., touch) so as to provide the biometric information more intuitively. According to various embodiments, the electronic device may analyze, based on various types of context information (e.g., a time, a place, a device usage log, etc.), biometric data collected by multiple electronic devices, store related insight information, and recognize a user context to provide coaching to the user through the insight information appropriate for corresponding context. According to various embodiments, utility (or usability) of the electronic device can be increased.

### BRIEF DESCRIPTION OF DRAWINGS

FIG. 1 illustrates an electronic device in a network environment according to an embodiment.

FIG. 2 is a block diagram illustrating the display device according to various embodiments.

FIG. 3 illustrates an example of a function processing module of an electronic device according to various embodiments.

FIG. 4 illustrates an example describing a health sensing model of an electronic device according to various embodiments.

FIG. 5 illustrates an example of estimating biometric information by an electronic device according to various embodiments.

FIG. 6 is a schematic diagram illustrating an example of providing biometric information based on a single electronic device according to various embodiments.

FIG. 7 is a schematic diagram illustrating an example of providing biometric information based on multiple electronic devices according to various embodiments.

FIG. 8 is a flowchart illustrating an operation method of an electronic device according to various embodiments.

FIG. 9 illustrates an example of a screen for providing biometric information by an electronic device according to various embodiments.

FIG. 10 illustrates an example of a screen for providing biometric information by an electronic device according to various embodiments.

FIG. 11 illustrates an example of a screen for providing biometric information by an electronic device according to various embodiments.

FIG. 12 is a flowchart illustrating an operation method of an electronic device according to various embodiments.

FIG. 13 is a flowchart illustrating an operation method of an electronic device according to various embodiments.

FIG. 14 is a flowchart illustrating an operation method of an electronic device according to various embodiments.

FIG. 15 is a flowchart illustrating an operation method of an electronic device according to various embodiments.

FIG. 16 is a flowchart illustrating an operation method of an electronic device according to various embodiments.

FIGS. 17A and 17B illustrate examples of screens for providing an insight by an electronic device according to various embodiments.

FIG. 18 is a flowchart illustrating an operation method of an electronic device according to various embodiments.

FIG. 19 illustrates an example of a screen for configuring a user-based place by an electronic device according to various embodiments.

FIG. 20 illustrates an example of a screen for configuring a user-based place by an electronic device according to various embodiments.

FIG. 21 illustrates an example of a screen for configuring a user-based place by an electronic device according to various embodiments.

FIG. 22 illustrates an example of a screen for configuring a user-based place by an electronic device according to various embodiments.

FIG. 23 illustrates an example of a screen for configuring a user-based place by an electronic device according to various embodiments.

FIG. 24 illustrates an example of a screen for providing biometric information by an electronic device according to various embodiments.

FIG. 25 illustrates an example of providing biometric information based on multiple electronic devices according to various embodiments.

FIG. 26 is a flowchart illustrating an operation method of an electronic device according to various embodiments.

FIG. 27 is a flowchart illustrating an operation method of an electronic device according to various embodiments.

FIG. 28 illustrates an example of a screen for providing biometric information according a place by an electronic device according to various embodiments.

FIG. 29 illustrates an example of a screen for providing biometric information according a place by an electronic device according to various embodiments.

FIG. 30 illustrates an example of a screen for providing biometric information according a place by an electronic device according to various embodiments.

### MODE FOR CARRYING OUT THE INVENTION

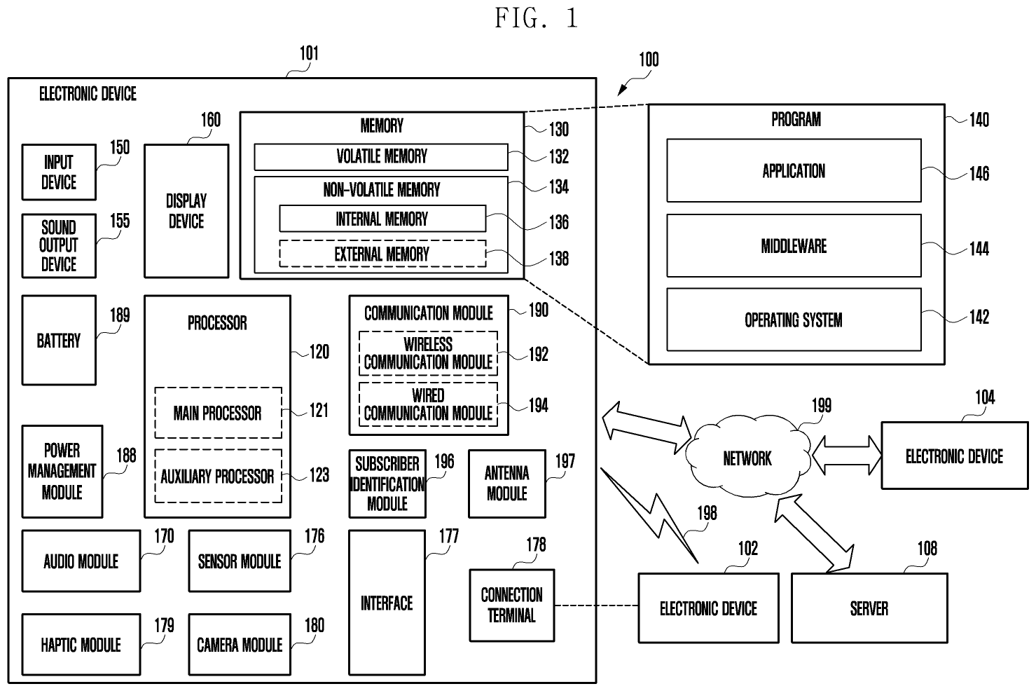

FIG. 1 illustrates an electronic device 101 in a network environment 100 according to an embodiment.

Referring to FIG. 1, the electronic device 101 in the network environment 100 may communicate with an electronic device 102 via a first network 198 (e.g., a short-range wireless communication network), with an electronic device 104 or a server 108 via a second network 199 (e.g., a long-range wireless communication network), or with the electronic device 104 via the server 108, and may include a processor 120, a memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) card 196, and an antenna module 197. At least one (e.g., the display device 160 or the camera module 180) of the components may be omitted from the electronic device 101, or one or more other components may be added in the electronic device 101. Some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. The processor 120 may load a command or data received from another component (e.g., the sensor module 176 or the communication module 190) in the volatile memory 132, process the command or the data stored in the volatile memory 132, and store resulting data in non-volatile memory 134. The processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). The auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101 and may include software (e.g., the program 140) and input data or output data for a command related thereto. The memory 130 may include the volatile memory 132 or the non-volatile memory 134.

The program 140 may be stored in the memory 130 as software, and may include an operating system (OS) 142, middleware 144, or an application 146.

The input device 150 may receive a command or data to be used by another component (e.g., the processor 120) of the electronic device 101, from the outside (e.g., a user) of the electronic device 101, and may include a microphone, a mouse, a keyboard, or a digital pen (e.g., a stylus pen).

The sound output device 155 may output sound signals to the outside of the electronic device 101 and may include a speaker or a receiver. The speaker may be used for general purposes, such as playing multimedia or playing record, and the receiver may be used for incoming calls and may be implemented as separate from, or as part of the speaker.

The display device 160 may visually provide information to the outside (e.g., a user) of the electronic device 101 and may include a display, a hologram device, or a projector and control circuitry to control a corresponding one of the display, hologram device, and projector. The display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa, and may obtain the sound via the input device 150, or output the sound via the sound output device 155 or a headphone of an external electronic device (e.g., an electronic device 102) directly (e.g., over wires) or wirelessly coupled with the electronic device 101.

The sensor module 176 may detect an operational state (e.g., power or temperature) of the electronic device 101 or an environmental state (e.g., a state of a user) external to the electronic device 101, and generate an electrical signal or data value corresponding to the detected state, and may include a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., over wires) or wirelessly, and may include a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102), and may include a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation, and may include a motor, a piezoelectric element, or an electric stimulator.

The camera module 180 may capture a still image or moving images and may include one or more lenses, image sensors, image signal processors, or flashes.

The power management module 188 may manage power supplied to the electronic device 101, and may be implemented as at least part of a power management integrated circuit (PMIC).

The battery 189 may supply power to at least one component of the electronic device 101, and may include a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. The communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., a LAN or a wide area network (WAN)). These various types of communication modules may be implemented as a single component (e.g., a single chip), or may be implemented as multi components (e.g., multi chips) separate from each other.

The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101 and may include an antenna including a radiating element composed of a conductive material or a conductive pattern formed in or on a substrate (e.g., a PCB). The antenna module 197 may include a plurality of antennas. In such a case, at least one antenna appropriate for a communication scheme used in the communication network, such as the first network 198 or the second network 199, may be selected by the communication module 190 (e.g., the wireless communication module 192) from the plurality of antennas. The signal or the power may then be transmitted or received between the communication module 190 and the external electronic device via the selected at least one antenna. Another component (e.g., an RFIC) other than the radiating element may be additionally formed as part of the antenna module 197.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

Commands or data may be transmitted or received between the electronic device 101 and the external electronic device 104 via the server 108 coupled with the second network 199. Each of the electronic devices 102 and 104 may be a device of a same type as, or a different type, from the electronic device 101.

All or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing, as at least part of a reply to the request. To that end, a cloud, distributed, or client-server computing technology may be used, for example.

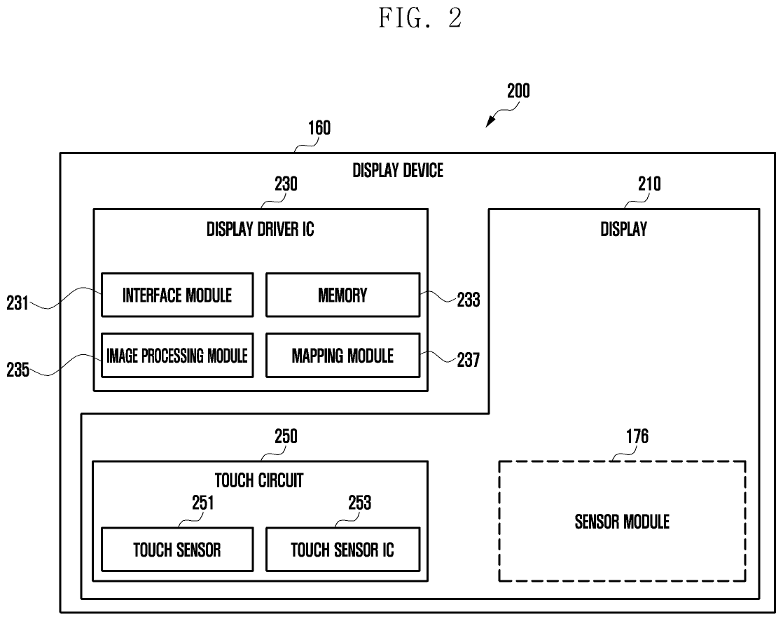

FIG. 2 is a block diagram 200 illustrating the display device 160 according to various embodiments.

Referring to FIG. 2, the display device 160 may include a display 210 and a display driver integrated circuit (DDI) 230 to control the display 210. The DDI 230 may include an interface module 231, memory 233 (e.g., buffer memory), an image processing module 235, or a mapping module 237.

The DDI 230 may receive image information that contains image data or an image control signal corresponding to a command to control the image data from another component of the electronic device 101 via the interface module 231. For example, according to an embodiment, the image information may be received from the processor 120 (e.g., the main processor 121 (e.g., an application processor)) or the auxiliary processor 123 (e.g., a graphics processing unit) operated independently from the function of the main processor 121. The DDI 230 may communicate, for example, with touch circuitry 350 or the sensor module 176 via the interface module 231. The DDI 230 may also store at least part of the received image information in the memory 233, for example, on a frame by frame basis.

The image processing module 235 may perform pre-processing or post-processing (e.g., adjustment of resolution, brightness, or size) with respect to at least part of the image data. According to an embodiment, the pre-processing or post-processing may be performed, for example, based at least in part on one or more characteristics of the image data or one or more characteristics of the display 210.

The mapping module 237 may generate a voltage value or a current value corresponding to the image data pre-processed or post-processed by the image processing module 235. According to an embodiment, the generating of the voltage value or current value may be performed, for example, based at least in part on one or more attributes of the pixels (e.g., an array, such as an RGB stripe or a pentile structure, of the pixels, or the size of each subpixel). At least some pixels of the display 210 may be driven, for example, based at least in part on the voltage value or the current value such that visual information (e.g., a text, an image, or an icon) corresponding to the image data may be displayed via the display 210.

According to an embodiment, the display device 160 may further include the touch circuitry 250. The touch circuitry 250 may include a touch sensor 251 and a touch sensor IC 253 to control the touch sensor 251. The touch sensor IC 253 may control the touch sensor 251 to sense a touch input or a hovering input with respect to a certain position on the display 210. To achieve this, for example, the touch sensor 251 may detect (e.g., measure) a change in a signal (e.g., a voltage, a quantity of light, a resistance, or a quantity of one or more electric charges) corresponding to the certain position on the display 210. The touch circuitry 250 may provide input information (e.g., a position, an area, a pressure, or a time) indicative of the touch input or the hovering input detected via the touch sensor 251 to the processor 120. According to an embodiment, at least part (e.g., the touch sensor IC 253) of the touch circuitry 250 may be formed as part of the display 210 or the DDI 230, or as part of another component (e.g., the auxiliary processor 123) disposed outside the display device 160.

According to an embodiment, the display device 160 may further include at least one sensor (e.g., a fingerprint sensor, an iris sensor, a pressure sensor, or an illuminance sensor) of the sensor module 176 or a control circuit for the at least one sensor. In such a case, the at least one sensor or the control circuit for the at least one sensor may be embedded in one portion of a component (e.g., the display 210, the DDI 230, or the touch circuitry 250)) of the display device 160. For example, when the sensor module 176 embedded in the display device 160 includes a biometric sensor (e.g., a fingerprint sensor), the biometric sensor may obtain biometric information (e.g., a fingerprint image) corresponding to a touch input received via a portion of the display 210. As another example, when the sensor module 176 embedded in the display device 160 includes a pressure sensor, the pressure sensor may obtain pressure information corresponding to a touch input received via a partial or whole area of the display 210. According to an embodiment, the touch sensor 251 or the sensor module 176 may be disposed between pixels in a pixel layer of the display 210, or over or under the pixel layer.

The electronic device 101 according to embodiments may be one of various types of electronic devices, such as a portable communication device (e.g., a smartphone), a computer device, a portable multimedia device, a portable medical device, a camera, a wearable device, or a home appliance. However, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise.

As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., over wires), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

In various embodiments, it is to be understood that if the term “measurement”, “collection”, or “sensing” is referred to, with or without the term “consecution” or “consecutively”, it means that a user may acquire minimum biometric data required for biometric information by using an electronic device 101, without the direct intent of the user for biometric measurement (e.g., executing an application related to biometric measurement and performing a series of operations related to biometric measurement), or through the direct intent of the user.

In various embodiments, biometric data (e.g., data related to biometric information) may indicate, for example, raw data, and may indicate data which an electronic device may receive through a biometric sensor of the electronic device. For example, the biometric data may include data before being processed (or data not being processed) to biometric information recognizable by the user.

In various embodiments, the term “place” may mean a spatial location (or a space) where a user stays. According to various embodiments, the “place” may include a fixed place (e.g., home, office 1, office 2, a park, etc.) based on a geographical location, and an unfixed place (e.g., car 1, car 2, etc.) based on a spatial location.

Various embodiments may display and provide the level of a user state (e.g., stress) according to a context by analyzing, based on at least one of a place, a stay time, or a usage log of the electronic device, biometric data (e.g., stress data) collected by the electronic device. Various embodiments may provide an insight (or an insight card) which can improve the level of the user state (e.g. stress), through usage log analysis of the electronic device.

According to various embodiments, the electronic device may store biometric data (e.g., stress, an HR, oxygen saturation, blood pressure, blood glucose, a step count, etc.) collected by the electronic device, together with place and time information, may display the level of the biometric data and a stay time according to a place, and may classify the level of the biometric data so that the classifications of the biometric data are displayed by colors. According to various embodiments, biometric data collected by a first electronic device (e.g., a wearable device) and a second electronic device (e.g., a smartphone) may be displayed together with time and place information, and an insight helpful for the user may be provided when the user enters the corresponding place or while the user stays at the corresponding place.

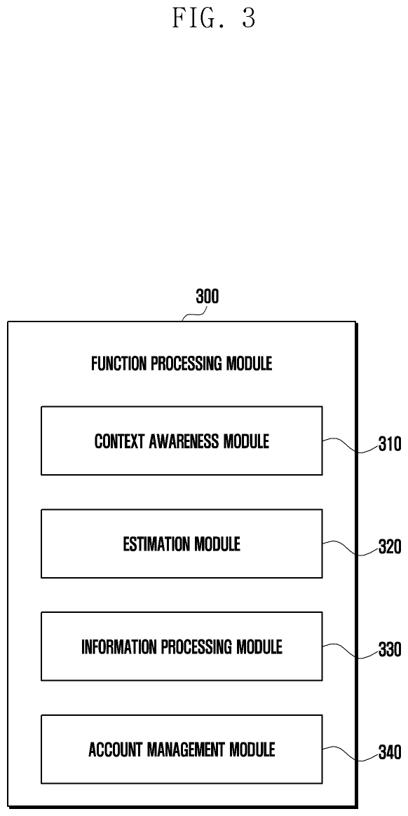

FIG. 3 illustrates an example of a function processing module of an electronic device according to various embodiments.

As shown in FIG. 3, according to various embodiments, FIG. 3 may show an example of a module (e.g., a function processing module 300) for executing a function related to estimating data related biometric information of a user, and providing the data to the user. For example, according to various embodiments, FIG. 3 may show an example of the function processing module 300 related to: collecting biometric data; providing biometric data for each place by classifying, based on a place (or stay time or a usage log), the collected biometric data; and providing coaching (or an insight) related to various states of the user. In various embodiments, the function processing module 300 may be included in a processor (e.g., the processor 120 of FIG. 1) including processing circuitry, as a hardware module or a software module.

Referring to FIG. 3, the function processing module 300 may include a context awareness module 310, an estimation module 320, an information processing module 330, and an account management module 340.

According to various embodiments, the context awareness module 310 may recognize various contexts related to the electronic device 101 (or a user) by using context awareness technology. The context awareness technology may indicate technology which abstracts various pieces of context information including a dynamic, an individual, or a static context occurring in a real space including the user, the electronic device 101, and an environment, inputs the various pieces of context information to a virtual space, and provides an intelligent service or customized information in accordance with the context of the user by utilizing the pieces of context information. According to an embodiment, the context awareness module 310 may recognize work, emotion, and a location of the user so that the electronic device 101 may recognize the context by itself even when there is no input from the user.

In various embodiments, the context awareness module 310 may analyze data (or information) input from various sensors (e.g., the sensor module 176 of FIG. 1) to determine the context. According to an embodiment, the context awareness module 310 may detect execution of an application (e.g., an application for performing a call operation, an application for playing music, an application for playing a video, an application related to a location-based service, or an application for an Internet service, etc.) in the electronic device 101. In response to the detection of the execution of the application, the context awareness module 310 may transmit, to the estimation module 320, information (e.g., a trigger signal) related to a start of measuring biometric information. In various embodiments, the context awareness module 310 may recognize various contexts such as a place where the user is located, a stay time, or the like, other than the individual context such as application execution, and may transmit, to the estimation module 320, information related to a start of measuring biometric information.

In various embodiments, the estimation module 320 may estimate a user state based at least on biometric data collected based on at least one sensor (e.g., the sensor module 176 of FIG. 1) and a context determined based on the context awareness module 310. According to an embodiment, the context awareness module 310 may monitor the at least one sensor and determine whether biometric information is acquired from the at least one sensor. According to an embodiment, the at least one sensor may include a sensor (e.g., an image sensor (or a camera module or an infrared camera), an iris (or retina) sensor, etc.) which may acquire a state (e.g., a face image and an eye image) of a user, or a biometric sensor (e.g., a fingerprint sensor, or electrode, etc.) which may directly acquire biometric information of the user.

In various embodiments, the biometric sensor may include, for example, a PPG sensor and an ECG sensor. According to an embodiment, the PPG sensor may emit infrared (IR) light or (red, green, or blue) visible light toward a body part, measure a signal reflected from the body part through a photodiode, and measure, based on a shape of a signal pattern or a change over time, a biometric state (e.g., a heart rate). According to an embodiment, the ECG sensor using an electrode, for example, may also measure the heart rate of the user in a different way from that of the PPG sensor. The electrode may be positioned at least a part of a front surface, a rear surface, or a side surface of the electronic device 101, and may be composed of a transparent electrode on a display 210 to enable biometric measurement on a screen.

According to various embodiments, biometric information may be measured by a camera (e.g., an image sensor). For example, when a front camera is activated, the camera may capture an image of a pattern of the bloodstream in a face, which the user may not see, and may measure a heart rate based the pattern.

In various embodiments, according to the type (or the shape) of the electronic device 101, the biometric sensor may be disposed at least one of a rear position of the electronic device 101, with which a part (e.g., the wrist, etc.) of the user's body may come into contact, a front position or a rear position of the electronic device 101, with which a finger of the user may come into contact when the user holds the electronic device 101, a position in the display device 160 at a front surface of the electronic device 101, or a side position of the electronic device 101. In various embodiments, the biometric sensor may include a sensor included in another electronic device (e.g., a wearable device, or an electronic device including a sensor), in addition to a sensor included in the electronic device 101. For example, when the electronic device 101 is a smartphone, the electronic device 101 may receive, through a communication module, biometric information measured through a wearable device worn on the user's body, and may provide the received biometric information to the user.

In various embodiments, the estimation module 320 may collect biometric data measured from the at least one sensor. The estimation module 320 may estimate, based on the collected biometric data, biometric information (e.g., information processed, based on biometric data, in a form recognizable by a user). For example, the estimation module 320 may estimate first biometric information (e.g., an HR and SPO2) based at least in part on the biometric data, and may estimate second biometric information (e.g., stress) based at least in part on the biometric data. For example, the estimation module 320 may estimate corresponding biometric information based on measurement conditions (or measurement times or measurement data amounts) required for the first biometric information and the second biometric information, respectively. According to an embodiment, the estimation module 320 may estimate biometric information by adding currently measured biometric data and integrating cumulatively stored biometric data. For example, the estimation module 320 may estimate biometric information by integrating inconsecutive measurement data related to the biometric information.

In various embodiments, the information processing module 330 (or a post-processing module) may perform post-processing to provide (or display) biometric information to a user. According to an embodiment, the information processing module 540 may link the estimated biometric information according to a place and select a region in which biometric information is to be represented. When corresponding biometric information is displayed, the information processing module 330 may perform post-processing so as to augment (or update) the displayed biometric information and provide the augmented (or updated) biometric information to the user.

According to various embodiments, the biometric sensor may include various sensors which may measure at least one of a physical change in body or a chemical change in body, and may include, for example, an optical sensor, an electronic signal measurement sensor, a pressure sensor, and the like.

According to various embodiments, the biometric sensor may include a health sensing model by which related biometric data may be acquired based on a signal measured from the user's body. For example, the biometric sensor may extract signals having various wavelengths from one PPG sensor, and, based on the signals, the biometric sensor may extract various pieces of biometric data according to the characteristics of reflection of an LED of each wavelength.

According to various embodiments, while a part of the user's body is in contact with the sensor of the electronic device 101 (or while the electronic device 101 is worn on a part of the user's body), the electronic device 101 may measure or collect biometric data of the user from the biometric sensor.

In various embodiments, biometric information measurable by the biometric sensor may include, for example, a heart rate (HR), a heart rate variation (HRV), oxygen saturation (SpO2), blood pressure (BP), blood glucose (BG), stress, emotion, skin hydration, or the like, as shown in [Table 1] below. According to various embodiments, the electronic device 101 (or a sensor of the electronic device 101) may include a health sensing model related to measurement of the above-described biometric information.

According to various embodiments, the biometric information measurable by the biometric sensor may vary as shown in [Table 1] below, and pieces of biometric information (e.g., measurement items) may have different conditions (e.g., a time required for measurement, or an amount of data required for measurement), and some pieces of biometric information may have the same condition or conditions similar to each other.

TABLE 1

| | | Minimum time |
|---|---|---|
| Measurement | | required for |
| Item | Description | measurement |
| Heart Rate | HR per minute—measurable by | 5-20 seconds |
| | PPG, ECG, and camera | |
| SpO₂ | Oxygen saturation—measurable | 5-20 seconds |
| | by PPG sensor | |
| | (multiple wavelengths) | |
| Heart Rate | Heart rate variation—measurable by | 5-20 seconds |
| Variation | PPG, ECG, and camera | |
| Blood pressure | Systolic pressure (SBP), | 30 seconds-1 |
| | diastolic pressure (DBP), | minute |
| | mean arterial pressure (MAP)—estimate | |
| | blood pressure | |
| | according to pulse waveform analysis | |
| | of PPG signal, or measurement of pulse | |
| | transition time by using multiple sensors | |
| Stress | Perform measurement based on | 20 seconds-1 |
| | HR and HRV of PPG | minute |
| | and ECG—accuracy enhanced when | |
| | information | |
| | such as blood pressure is added | |
| Blood Glucose | Measure concentration of glucose in | 30 seconds-1 |
| | blood—measurable by PPG sensor | minute |
| Body | Quantitatively provide body | 5-20 seconds |
| Composition | composition—measurable by | |
| | electrode | |
| | (bioelectrical impedance analysis) | |
| Skin | Detect skin tone, wrinkles, | Within 5 |
| | erythema, and acne by | seconds |
| | using camera | |
| Emotion | Measure emotional state by | 1 minute or |
| | analyzing information | longer |
| | measured by sensor | |
| | (PPG, ECG, and the like) and | |
| | facial feature point acquired by camera | |

According to various embodiments, the account management module 340 may configure and/or manage a user account. According to an embodiment, the account management module 340 may access a server by using the user account, and may provide information related to the user account received from the server. According to an embodiment, the account management module 340 may configure, for a server 603, various pieces of information (e.g., personal user information) relating to a user by using the user account. According to an embodiment, the personal user information may include, for example, profile information relating to a user profile, device information relating to a user device (or an electronic device), health information relating to a user's health, place information relating to a place registered by a user, application information relating to an application, or the like.

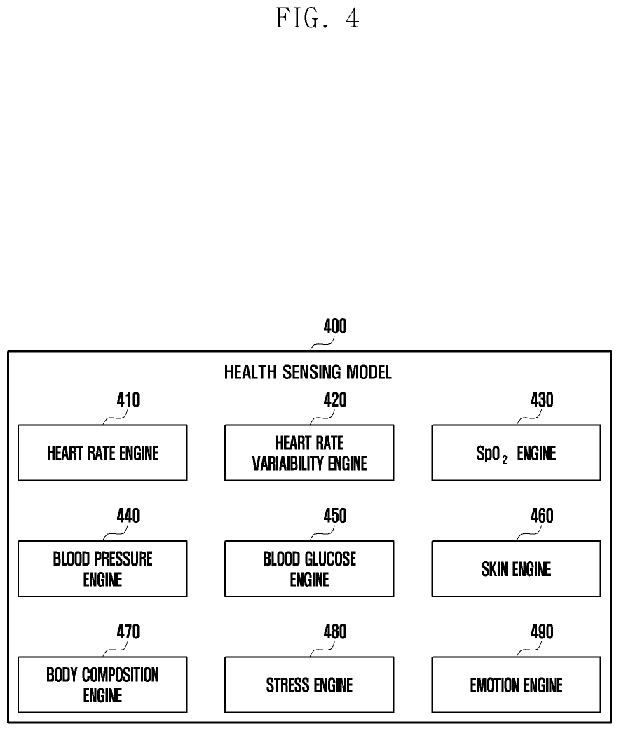

FIG. 4 illustrates an example describing a health sensing model of an electronic device according to various embodiments.

Referring to FIG. 4, a health sensing model 400 of a biometric sensor (e.g., an optical sensor) may include, for example, a heart rate engine 410, a heart rate variability engine 420, an oxygen saturation engine (SpO2 engine) 430, a blood pressure engine 440, a blood glucose engine 450, a skin engine 460, a body composition engine 470, a stress engine 480, and an emotion engine 490.

In various embodiments, an example of measuring biometric data based on the health sensing model 400 of the biometric sensor (e.g., an optical sensor or a PPG sensor) is described below.

According to an embodiment, the heart rate engine 410 and the heart rate variation engine 420 may measure the heart rate and the heart rate variation (HR/HRV) according to a signal measured by the biometric sensor. According to an embodiment, the SpO2 engine 430 may measure the SpO2 by using the biometric sensor which may perform measurement in two or more wavelengths.

According to an embodiment, the blood pressure engine 440 may estimate the blood pressure (BP) according to a pulse wave analysis (PWA) of a signal measured by the biometric sensor. For example, the blood pressure engine 440 may estimate the blood pressure (BP) by extracting feature points from the measured waveform and substituting a corresponding feature point value to a predetermined model (e.g., the pressure engine 440). In addition, according to an embodiment, the blood pressure engine 440 may estimate the blood pressure (BP) by measuring a subtle change in facial color from an image acquired from a camera (e.g., a front camera), extracting a waveform in real time, and measuring a transition time (e.g., a pulse transition time (PTT)) difference from the signal measured by the biometric sensor. Further, the blood pressure engine 440 may estimate the blood pressure (BP) by using the above-described two ways.

According to an embodiment, the blood glucose engine 450 may estimate the blood glucose (BG) based on a change in the concentration of glucose in the blood by extracting a feature point and the absorbance of a signal measured by the biometric sensor which may perform measurement in two or more wavelengths.

According to an embodiment, the skin engine 460 may provide, in real time, quantitative information relating to skin such as a skin tone, wrinkles, erythema, acne, or the like by analyzing a face image (e.g., a selfie image) of a user, acquired from a camera (e.g., a front camera).

According to an embodiment, the body composition engine 470 may estimate body composition (e.g., body water, body fat, muscle mass, etc.) by analyzing bioelectrical impedance measured from an electrode. For example, when current passes through various parts of the body, a voltage may drop, wherein the body composition engine 470 may acquire indirect information relating to a physical feature of the corresponding part through the measured degree of the voltage drop, and may quantify body water, fat mass, and the like therefrom.

According to an embodiment, the stress engine 480 may analyze an aspect of a change during a predetermined time by using the HR/HRV measured in advance, and may estimate the stress. According to an embodiment, BP information may also be reflected in the aspect of the change during a predetermined time, whereby stress estimation accuracy may be enhanced.

According to an embodiment, the emotion engine 490 may estimate and quantify the emotion related to the user, such as happiness, sadness, anger, excitement, or the like, from a predetermined model by extracting, in addition to the measured biometric data, a feature of a user's facial expression from an image (e.g., a selfie image) acquired from a camera. According to an embodiment, the emotion engine 490 may also detect specific emotion (e.g., anxiety, excitement, etc.) of the user by using measurement information relating to the stress and/or the heart rate.

According to various embodiments, the electronic device 101 may estimate biometric data while the user's body is in contact with the biometric sensor. According to an embodiment, the electronic device 101 may process biometric information measurable for a first time (e.g., a short time) first, and then may sequentially process augmented biometric information later. For example, the heart rate (HR) or the oxygen saturation (SpO2) may be estimated for a short time (e.g., approximately 5-20 seconds). For example, when the biometric data is further measured for a second time (e.g., a time longer than the first time) beyond the first time, for example, the heart rate variation (HRV), the blood pressure (BP), the blood glucose (BG), or the like may be sequentially estimated in time sequence (or most of multiple pieces of information are measured simultaneously). For example, when the biometric data is measured for a third time (a time longer than the second time) beyond the second time, emotion information may be estimated for example.

Hereinafter, an example of estimating biometric information according to a trigger (or an event) related to estimation of biometric information by using the health sensing model as described in the above-described example in various embodiments is described below.

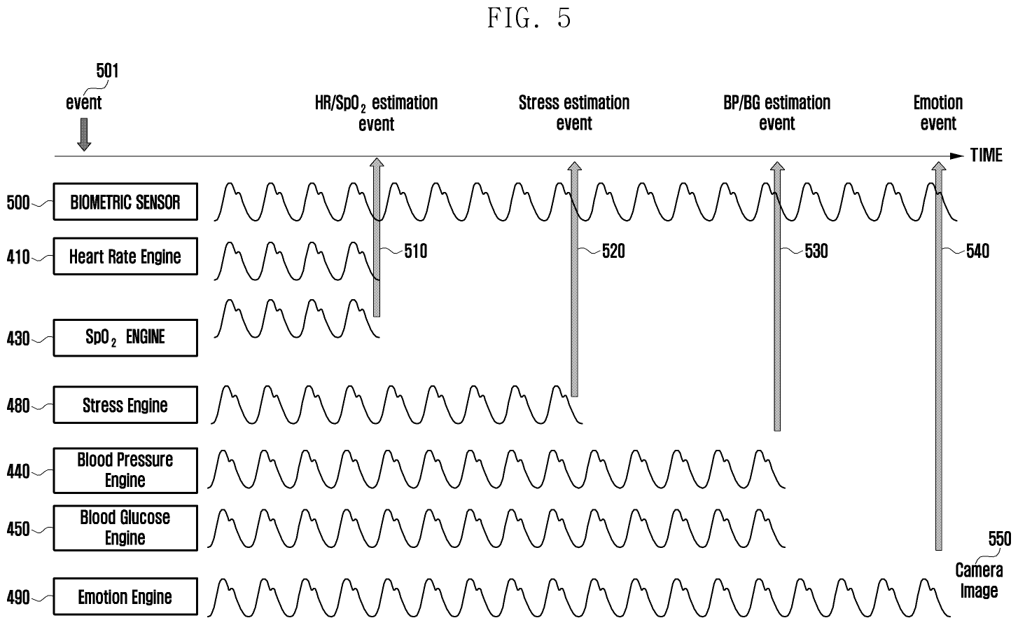

FIG. 5 illustrates an example of estimating biometric information by an electronic device according to various embodiments.

Referring to FIG. 5, FIG. 5 may show an example describing occurrence of a measurement event based on the health sensing model 400 illustrated in FIG. 4.

According to an embodiment, the electronic device 101 may extract M pieces of biometric information (e.g., a heart rate (HR), stress, blood glucose (BG), blood pressure (BP), emotion, etc.) from N biometric sensors (e.g., a PPG sensor, an electrode, an image sensor (e.g., a camera), an accelerometer sensor, etc.). According to an embodiment, there may be multiple pieces of biometric information measurable by using one biometric sensor, and, for example, M, the number of pieces of biometric information, may include a number equal to or larger than N, the number of biometric sensors (e.g., M≥N).

According to various embodiments, the electronic device 101 may simultaneously extract various models, and to this end, various engines (e.g., the heart beat engine 410, the blood pressure engine 440, the blood glucose engine 450, the stress engine 480, the emotion engine 490, etc.) may simultaneously operate. According to various embodiments, an input signal of each engine may be identical, but result events may be transmitted at different timings since a processing engine independently operates. For example, there may be a single input signal (e.g., an event 501) input through a biometric sensor 500 (e.g., a PPG sensor), and there may be multiple engines (e.g., the heart beat engine 410, the oxygen saturation (SpO2) engine 430, the stress engine 480, the blood pressure engine 440, the blood glucose engine 450, the emotion engine 490, etc.) operable based on the input signal by the biometric sensor 500. According to various embodiments, the multiple engines may independently operate, and a measurement event may occur at each timing based at least on a reference time (or a minimum time) required to estimate corresponding biometric information.

As shown in FIG. 5, according to an embodiment, the electronic device 101 may first generate, based on the heart rate engine 410 at a first timing 510, an event related to heart rate (HR) information. According to various embodiments, since the heart rate information needs to be continuously monitored, the heart rate engine 410 may continuously transmit a related event to the processor 120 even after the initial event. According to an embodiment, the heart rate variation and the oxygen saturation may mostly have the same measurement time as (or a measurement time similar to) that of the heart rate information. According to an embodiment, mostly, an event related to oxygen saturation information and an event related to heart rate variation information may simultaneously occur at the first timing 510 by the oxygen saturation engine 430 and by the heart rate variation engine 420 (not shown in FIG. 5), respectively. According to an embodiment, the longer the heart rate variation is observed, the more accurate the heart rate variation becomes, and thus, an event transition timing of the heart rate variation may be later than that of the heart rate. For example, the more the biometric data are collected for calculation, the more accurate the heart rate variation may be, and thus a determination timing for providing appropriated reliability of the heart rate variation may be later than that of the heart rate.

According to an embodiment, the electronic device 101 may generate an event related to stress information by using the stress engine 480 at a second timing 520. According to various embodiments, the stress engine 480 may measure the stress based on the heart rate variation. According to an embodiment, when outputting (e.g., displaying) information (or an object) related to the biometric information, the electronic device 101 may not display an accurate number (e.g., a quantitative value) relating to corresponding biometric information, but may provide a trend (e.g., a qualitative value) on biometric information for use of guiding (or coaching) the corresponding biometric information to a user. For example, in the case of the guidance of the biometric information, since sensitivity of the user with respect to receiving accuracy or reliability of the biometric information may be low, the corresponding biometric information may be displayed even though the measurement time of the biometric information is short.

According to an embodiment, the electronic device 101 may generate an event related to blood pressure information by using the blood pressure engine 440 at a third timing 530. According to an embodiment, the electronic device 101 may also generate an event related to blood glucose information by using the blood glucose engine 450 at the third timing 530. According to an embodiment, in the case of the blood pressure, it may be important to extract the optimal signal waveform, and to this end, “representativity” or “statistical reliability” of the waveform needs to be increased through acquisition of multiple waveforms, and according to whether the “representativity” or “statistical reliability” of the waveform is increased, a measurement time (e.g., an event generation timing) may become very short or very long.

According to an embodiment, the electronic device 101 may generate an event related to emotion information by using the emotion engine 490 at a fourth timing 540. According to an embodiment, since the emotion may be associated with the stress, an event may be provided based on a stress value. According to an embodiment, the electronic device 101 may combine voice or facial expression information of the user to express complex and precise emotion rather than a fragmentary emotional state. For example, in a selfie mode, image information (e.g., a camera image 550) may be acquired through an image sensor (e.g., a camera), an emotional state of the user may be determined based on the acquired image information and biometric information (e.g., stress information) by the biometric sensor 500, and an emotional state of the user may be determined based on biometric information and voice information during a call.

According to various embodiments, the various measurement engines related to biometric information measurement of the user, as shown in FIGS. 4 and 5, may have different measurement types and conditions (e.g., a time required for measurement, etc.) required for measurement of each of the measurement engines. Accordingly, in FIG. 5, a measurement event relating to each of the measurement engines may be generated in a sequence satisfying (having) a condition for each of the measurement engines, and a time point at which a measurement event occurs may be also different according to a context.

According to an embodiment, at the time of measurement, the more the sampling data, accuracy of the heart rate or the heart rate variation may be enhanced, and thus, a scheme requiring a predetermined time may be included. According to an embodiment, in the case of the oxygen saturation (SpO2), a scheme of detecting changes in both types of light of an infrared (IR) light and red light may be included. For example, in the case of the heart rate, the heart rate variation, or the oxygen saturation, there may be a default time for sequential measurement. According to an embodiment, a scheme of measuring the blood pressure or the blood glucose requires one complete (or clean (e.g., noise-free)) waveform, but the one complete waveform may not be acquired at once according to a measurement context. As described above, a minimum time and a maximum time required for measurement for each of the measurement engines may be different according to a context. Accordingly, a measurement event related to each measurement engine may occur differently according to a measurement environment or various contexts such as matching with the signal waveform measured in advance.

In FIG. 5 above, an example of measuring biometric data and generating each measurement event related to biometric information at a corresponding timing has been described. According to various embodiments, biometric data (e.g., raw data or source data) acquired (or measured) through the biometric sensor may be accumulated (or stored) and managed, and when the measurement event occurs, a timing to generate the measurement event of the related biometric information may be shortened by using the accumulated data. For example, according to various embodiments, an event related to the biometric information may be generated by integration of inconsecutive measurement data.

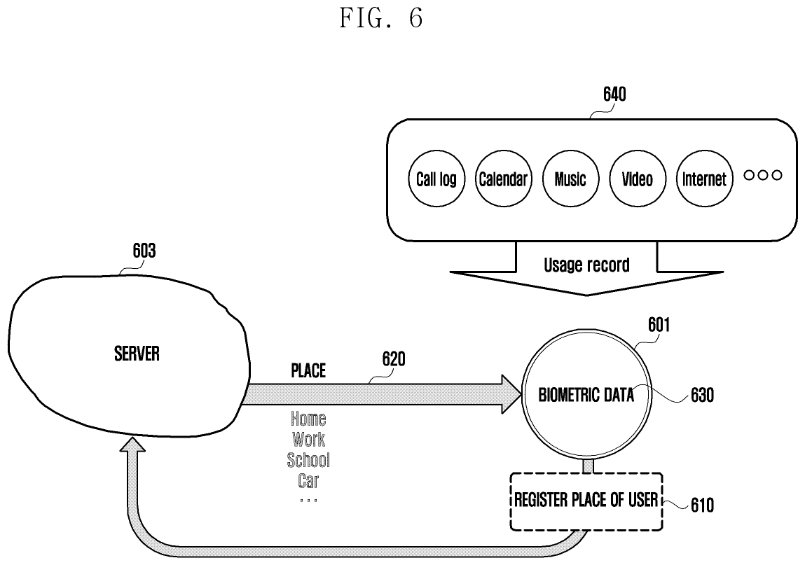

FIG. 6 is a schematic diagram illustrating an example of providing biometric information based on a single electronic device according to various embodiments.

As shown in FIG. 6, FIG. 6 may show an example of providing biometric information related to a user by using the single electronic device 101. In FIG. 6, the electronic device 101 is exemplified by a wearable device 601, and an example of providing biometric information based on a place of the user by the wearable device 601 is described, but is not limited thereto, and the operation corresponding to FIG. 6 may be operated by an electronic device such as a smartphone. According to various embodiments, the wearable device 601 may include some or all of the electronic device 101 of FIG. 1.

According to an embodiment, a server 603 may indicate a server for controlling and managing various pieces of information (e.g., personal user information) related to a user, by using a user account. For example, the server 603 may include an account server. According to various embodiments, the various pieces of information relating to the user correspond to information registered to the server 603 by the user by using the user account, and may include, for example, profile information relating to a user profile, device information relating to a user device (or an electronic device), health information relating to a user's health, place information relating to a place registered by a user, application information relating to an application, or the like.

According to an embodiment, the wearable device 601 may be worn on the user's body, and may constantly measure biometric data of the user in a state in which the wearable device 601 is worn on the user's body. The wearable device 601 may provide related biometric information based at least on the measured biometric data to the user.

According to various embodiments, the wearable device 601 may communicate with the server 603 by using a communication module (e.g., the communication module 190 of FIG. 1), and may access the server 603 by using a user account. According to an embodiment, the user may communicatively connect the wearable device 601 to the server 603 in a configured communication scheme, and may log in to the server 603 by using the user account.

The wearable device 601 may access the server 603 and display an interface (or a screen) related to a user account configuration through a display device (e.g., the display device 160 of FIG. 1). The user may configure and register a user-defined (or user's favorite) place to the server 603 by using the displayed interface. According to various embodiments, the place means a spatial location (or a space) where a user stays, and, for example, the place may include a fixed place (e.g., home, office 1, office 2, a park, etc.) based on a geographical location, and an unfixed place (e.g., car 1, car 2, etc.) based on a spatial location.

Referring to FIG. 6, the operation of providing biometric information by the wearable device 601 according to various embodiments is described below.

As shown by reference numeral 610, the user may register a user's place to the server 603 by using the wearable device 601.

As shown by reference numeral 620, the server 603 may synchronize place information (e.g., location information) relating to the place registered to the server 603 by the user with the wearable device 601. According to an embodiment, the server 603 may periodically transmit (e.g., in a push scheme) the place information to the wearable device 601. For example, the server 603 may automatically transmit the place information on the server 603 to the wearable device 601 by the operation of the server 603, without depending on the wearable device 601. According to an embodiment, the server 603 may transmit (e.g., in a pull scheme) the place information to the wearable device 601 in response to a request from the wearable device 601. For example, the server 603 may transmit the place information on the server 603 to the wearable device 601 in response to the access of the wearable device 601 to the server 603 by using the user account, or in response to the access and the request for the place information.

As shown by reference numeral 630, the wearable device 601 may acquire (or sense) biometric data related to the user and store the acquired biometric data. According to various embodiments, when storing the biometric data, the wearable device 601 may store the biometric data together with (or by matching with) place information (or including time information as well) relating to a place where the biometric data is acquired.

According to various embodiments, when a request to display biometric information from the user is detected, or when at least one piece of biometric information may be generated based on biometric data, the wearable device 601 may display the biometric information through a display device (e.g., the display device 160 of FIG. 1) and provide the biometric information to the user. According to various embodiments, when providing the biometric information, the wearable device 601 may provide the biometric information by classifying the biometric information according to a duration for each place. According to an embodiment, the wearable device 601 may provide the biometric information by classifying the biometric information according to a measured time and/or place, (or according to information on when/where the biometric information is measured), thereby allowing the user to recognize when/where the corresponding result is measured.

According to various embodiments, when the wearable device 601 detects an entrance into a specific place (e.g., home, an office, a car, etc.) related to the user, the wearable device 601 may provide, based on previous biometric information (e.g., stress information) at the corresponding place, an insight related to the user. According to various embodiments, the operation of recognizing a user context and providing an insight appropriate for the corresponding context will be described in detail with reference to the drawings below.

As shown by reference numeral 640, the wearable device 601 may recognize (e.g., perform context awareness) and record various usage logs related to the use of the wearable device 601 by the user. According to an embodiment, the wearable device 601 may monitor and record an application (e.g., an application such as Call, Calendar, Music, Video, or Internet) used by the user by using the wearable device 601, or contents (e.g., a call log, a schedule, a music playlist (or item), a video playlist (or item), a web browsing history, etc.) used through the application. According to an embodiment, when monitoring the usage log, the wearable device 601 may acquire biometric data of the user, and may store biometric data (or biometric information by biometric data) together with (or in association with or by mapping with) the corresponding usage log.

According to various embodiments, the wearable device 601 may determine whether a user context requires an insight, based on consecutive measurement biometric data for biometric information and an average amount of changes in the biometric information. According to an embodiment, when it is determined, based on the consecutive measurement biometric data for stress information and the average amount of changes in the stress information (or a stress index), that the user context indicates that the user needs to calm his or her mind (e.g., when the stress index is greater than a reference stress index), the wearable device 601 may provide an appropriate insight. According to various embodiments, the wearable device 601 may provide, to the user, an insight for helping the user know how to handle contexts in which negative stress occurs.

According to an embodiment, the wearable device 601 may recommend, to the user, an object positively affecting the user. According to an embodiment, the wearable device 601 may provide an insight (or recommendation or tips) for inducing the user to attempt to make a call to another user (e.g., a family member, a friend, or a person who lowered the stress index of the user when the user talks on the phone with the same person) positively affecting the user. According to an embodiment, the wearable device 601 may provide an insight (or recommendation or tips) for inducing the user to use an item (e.g., an application, a content, an event, etc.) positively affecting the user. In various embodiments, the operation of recognizing a user context and providing an insight appropriate for the corresponding context will be described in detail with reference to the drawings below.

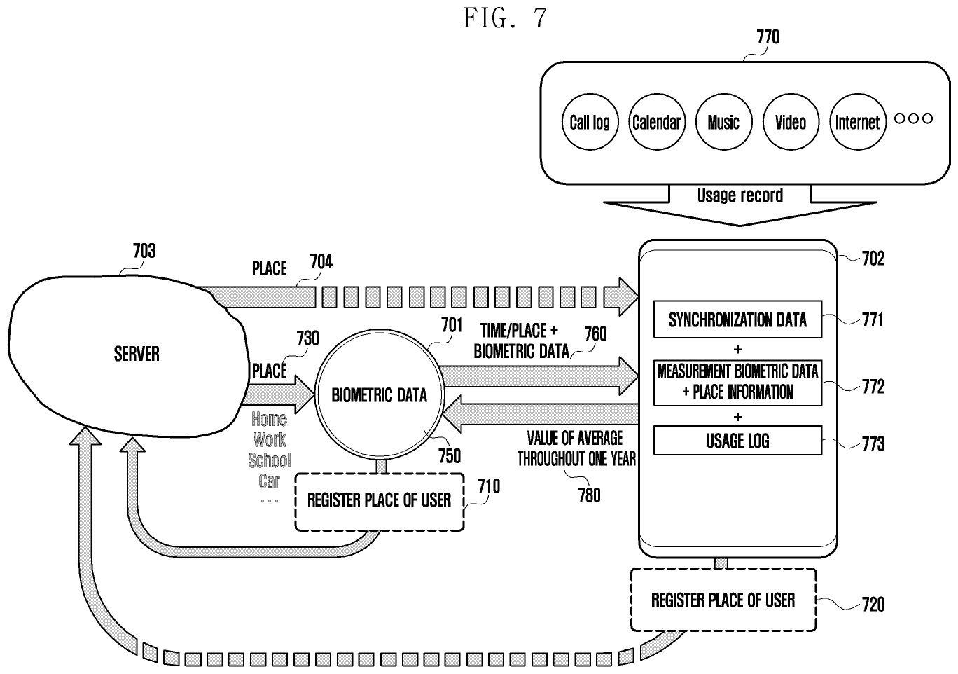

FIG. 7 is a schematic diagram illustrating an example of providing biometric information based on multiple electronic devices according to various embodiments.

As shown in FIG. 7, an example of providing biometric information related to a user, based on interworking of multiple electronic devices (e.g., a first electronic device 701 and a second electronic device 702) is described in FIG. 7. For example, in FIG. 7, the first electronic device 701 is exemplified by a wearable device and the second electronic device 702 is exemplified by a smartphone, wherein it is exemplified that the first electronic device 701 measures biometric data based on a place of a user, and the second electronic device 702 provides biometric information based on a place of a user. According to various embodiments, the first electronic device 701 and the second electronic device 702 may include some of all of the electronic device 101 of FIG. 1.

According to an embodiment, a server 703 may indicate a server for controlling and managing various pieces of information (e.g., personal user information) related to a user by using a user account. For example, the server 703 may include an account server. According to various embodiments, the various pieces of information relating to the user may include information (profile information, device information, health information, place information, or application information, etc.) registered to the server 703 by the user, using the user account.

According to an embodiment, the first electronic device 701 may include some of all of operations executed in the wearable device 601, described above with reference to FIG. 6. For example, the first electronic device 701 may constantly measure biometric data of the user in a state in which the first electronic device 701 is worn on the user's body. The first electronic device 701 may provide related biometric information based at least on the measured biometric data to the user.

According to various embodiments, the first electronic device 701 may communicate with the server 703 by using a communication module (e.g., the communication module 190 of FIG. 1), and may access the server 703 by using the user account. According to an embodiment, the first electronic device 701 may access the server 703 by using the user account and display an interface (or a screen) related to a user account configuration through a display device (e.g., the display device 160 of FIG. 1). The first electronic device 701 may configure and register a user-defined (or user's favorite) place to the server 703. According to various embodiments, the place means a spatial location (or a space) where a user stays, and, for example, the place may include a fixed place (e.g., home, office 1, office 2, a park, etc.) based on a geographical location, and an unfixed place (e.g., car 1, car 2, etc.) based on a spatial location.

According to various embodiments, the second electronic device 702 may communicate with the server 703 by using a communication module (e.g., the communication module 190 of FIG. 1) and may access the server 703 by using the user account. According to an embodiment, the second electronic device 702 may access the server 703 by using the user account and display an interface (or a screen) related to a user account configuration through a display device (e.g., the display device 160 of FIG. 1). The second electronic device 702 may configure and register a user-defined (or user's favorite) place to the server 703.

According to various embodiments, the user-defined place configured and registered to the server 703 by each of the first electronic device 701 or the second electronic device 702 may be managed by using the user account through the server 703, wherein the place managed by using the user account may be synchronized with the first electronic device 701 and the second electronic device 702 through the server 703.

Referring to FIG. 7, the operation of providing biometric information based on interworking of the first electronic device 701 and the second electronic device 702 according to various embodiments is described below.

As shown by reference numeral 710 and reference numeral 720, the user may register the user's place to the server 703 by using at least one of the first electronic device 701 or the second electronic device 702.

As shown by reference numeral 730 and reference numeral 740, the server 703 may synchronize place information (e.g., location information) relating to the place registered by the user with the first electronic device 701 and the second electronic device 702. According to an embodiment, the server 703 may periodically transmit (e.g., in a push scheme) the place information to the first electronic device 701 and the second electronic device 702. For example, the server 703 may automatically transmit the place information on the server 703 to the first electronic device 701 and/or the second electronic device 702 by the operation of the server 703, without depending on the first electronic device 701 or the second electronic device 702.

According to an embodiment, the server 703 may transmit (e.g., in a pull scheme) the place information to the first electronic device 701 or the second electronic device 702 in response to a request from the first electronic device 701 or the second electronic device 702. For example, the server 703 may transmit the place information on the server 703 to the corresponding electronic device in response to the access of the first electronic device 701 or the second electronic device 702 to the server 703 by using the user account, or in response to the access and the request for the place information. When a new place is added to the server 703 by the first electronic device 701 or the second electronic device 702, the server 703 may synchronize the new place with another electronic device by using the user account.

As shown by reference numeral 750, the first electronic device 701 may acquire (or sense) biometric data related to the user and store the acquired biometric data. According to various embodiments, when storing the biometric data, the first electronic device 701 may store the biometric data together with (or by matching with) place information (or including time information as well) relating to a place where the biometric data is acquired.

As shown by reference numeral 760, the first electronic device 701 may transmit (or share) the stored data to (or with) the second electronic device 702. For example, the first electronic device 701 may transmit place information and consecutively measured biometric data (or consecutive measurement data) to the second electronic device 702. According to an embodiment, the first electronic device 701 may acquire consecutive measurement data and transmit the consecutive measurement data and place information (or including time information as well) to the second electronic device 702 whenever the consecutive measurement data is acquired. According to an embodiment, the first electronic device 701 may transmit the consecutive measurement data and place information (or including time information as well) to the second electronic device 702 when the consecutive measurement data is acquired at a configured place.

According to various embodiments, when a request to display biometric information from the user is detected, or when at least one piece of biometric information may be generated based on biometric data, the first electronic device 701 may display the biometric information through a display device (e.g., the display device 160 of FIG. 1) and provide the biometric information to the user. According to various embodiments, when providing the biometric information, the first electronic device 701 may provide the biometric information by classifying the biometric information according to a duration for each place. According to an embodiment, the first electronic device 701 may provide the biometric information by classifying the biometric information according to a measured time and/or place, (or according to information on when/where the biometric information is measured), thereby allowing the user to recognize when/where the corresponding result is measured.

According to various embodiments, when the first electronic device 701 detects an entrance into a specific place (e.g., home, an office, a car, etc.) related to the user, the first electronic device 701 may provide, based on previous biometric information (e.g., stress information) at the corresponding place, an insight related to the user. According to various embodiments, the operation of recognizing a user context and providing an insight appropriate for the corresponding context will be described in detail with reference to the drawings below.

According to various embodiments, when a request to display biometric information from the user is detected, when biometric information is received from the first electronic device 701, or when at least one piece of biometric information may be generated based on the biometric data received from the first electronic device 701, the second electronic device 702 may display the biometric information through a display device (e.g., the display device 160 of FIG. 1) and provide the biometric information to the user.

According to various embodiments, when providing biometric information, the second electronic device 702 may provide the biometric information based at least on data 771 (e.g., synchronization data) acquired from the first electronic device 701, biometric data 772 measured by the second electronic device 702, or various usage logs 773 related to the use of the electronic device 702.

According to various embodiments, when providing the biometric information, the second electronic device 702 may provide the biometric information by classifying the biometric information according to a duration for each place. According to an embodiment, the second electronic device 702 may provide the biometric information by classifying the biometric information according to a measured time and/or place, thereby allowing the user to recognize when/where the corresponding result is measured.

According to various embodiments, when the second electronic device 702 detects an entrance into a specific place (e.g., home, an office, a car, etc.) related to the user, the second electronic device 702 may provide, based on previous biometric information (e.g., stress information) at the corresponding place, an insight related to the user. According to various embodiments, the operation of recognizing a user context and providing an insight appropriate for the corresponding context will be described in detail with reference to the drawings below.

As shown by reference numeral 770, the second electronic device 702 may recognize (e.g., perform context awareness) and record various usage logs related to the use of the second electronic device 702 by the user. According to an embodiment, the second electronic device 702 may monitor and record an application (e.g., an application such as Call, Calendar, Music, Video, or Internet) used by the user by using the second electronic device 702, or contents (e.g., a call log, a schedule, a music playlist (or item), a video playlist (or item), a web browsing history, etc.) used through the application. According to an embodiment, when monitoring the usage log, the second electronic device 702 may acquire biometric data of the user, and may store biometric data (or biometric information by biometric data) together with (or in association with or by mapping with) the corresponding usage log.

According to various embodiments, at least one of the first electronic device 701 or the second electronic device 702 may determine whether a user context requires an insight, based on consecutive measurement biometric data for biometric information and an average amount of changes in the biometric information. According to an embodiment, when it is determined, based on the consecutive measurement biometric data for stress information and the average amount of changes in the stress information (or a stress index), that the user context indicates that the user needs to calm his or her mind (e.g., when the stress index is greater than a reference stress index), at least one of the first electronic device 701 or the second electronic device 702 may provide an appropriate insight. According to various embodiments, at least one of the first electronic device 701 or the second electronic device 702 may provide, to the user, an insight for helping the user know how to handle contexts in which negative stress occurs.

According to an embodiment, at least one of the first electronic device 701 or the second electronic device 702 may recommend, to the user, an object positively affecting the user. According to an embodiment, at least one of the first electronic device 701 or the second electronic device 702 may provide an insight for inducing the user to attempt to make a call to another user (e.g., a family member, a friend, or a person who lowered the stress index of the user when the user talks on the phone with the same person) positively affecting the user. According to an embodiment, at least one of the first electronic device 701 or the second electronic device 702 may provide an insight (or recommendation or tips) for inducing the user to use an item (e.g., an application, contents, an event, etc.) positively affecting the user. In various embodiments, the operation of recognizing a user context and providing an insight appropriate for the corresponding context will be described in detail with reference to the drawings below.

As described above, an electronic device 101 according to various embodiments may include: a sensor module 176, a display 160, and a processor 120, wherein the processor 120 is configured to: acquire, using the sensor module 176, biometric information of a user and place information related to the user; match the biometric information with the place information; display an interface including biometric information for a predetermined period of time through the display device 160; determine a place of a region selected by the user and a duration corresponding to the place in the interface; and specify the duration and display biometric information by highlighting the biometric information within the duration in the interface.

According to various embodiments, the processor 120 may analyze a usage log of the electronic device 101, and may match the usage log with biometric information related to the usage log.

According to various embodiments, the processor 120 may determine, based on the biometric information and the place information, a user context, and may output an insight related to the user context.

According to various embodiments, the processor 120 may determine, based on the biometric information and the usage log, a user context, and may output an insight related to the user context.

According to various embodiments, the processor 120 may determine a user context based at least on the biometric information, the place information, or the usage log, and may output an insight related to the user context when the user context is included in a configured condition.

According to various embodiments, the processor 120 may estimate, based on biometric information in a specific context related to a user, a user state, may generate, based on the user state, context data related to the user context, and may store the context data.

According to various embodiments, the processor 120 may analyze biometric information, may determine whether a user state according to the biometric information is included in a configured condition, may extract an insight related to the user state when the user state is included in the configured condition; and may output the insight.

According to various embodiments, the processor 120 may perform context awareness when biometric information is collected, and may output, based on context information according to the context awareness and a user state according to the biometric information, a related insight.

According to various embodiments, the place information related to the user may include information registered to a server by using a user account.

According to various embodiments, the processor 120 may classify biometric information according to a place, and display place-specific averages of biometric information for a predetermined period of time by colors, through the interface.

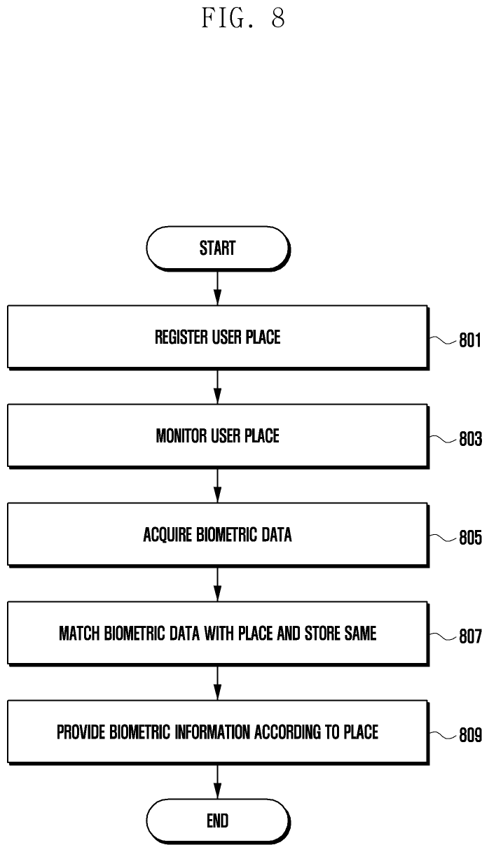

FIG. 8 is a flowchart illustrating an operation method of an electronic device according to various embodiments.

Referring to FIG. 8, in operation 801, a processor 120 (e.g., at least one processor including processing circuitry) (or the function processing module 300 of FIG. 3) of an electronic device 101 may register a user place. According to an embodiment, the processor 120 may access a server (e.g., an account server) by using a user account in response to a user input. The processor 120 may display an account screen received from the server, through the display device 160, in response to the access to the server by using the user account. In an account screen, the processor 120 may receive an input of a user place, transmit the input user place to the server, and configure and register place information to the user account through the server. According to an embodiment, when registering the user place to the user account, the processor 120 may also store related place information to the electronic device 101. According to an embodiment, when the processor 120 has logged in to the user account or is in a logged-in state in the electronic device 101, the processor 120 may identify place information relating to the user place from the user account.

In operation 803, the processor 120 may monitor the user place. According an embodiment, the processor 120 may perform context awareness by using various sensors of the electronic device 101, and may determine a place where the electronic device 101 (or a user) stays, as one of the context awareness. According to an embodiment, the processor 120 may monitor whether a current location of the electronic device 101 corresponds to place 1 (e.g., home), place 2 (e.g., an office), or place 3 (e.g., a car) based at least on location information, an amount of changes in the location information, acceleration information, movement information, or the like.

In operation 805, the processor 120 may acquire biometric data of the user. According to an embodiment, the processor 120 may cause a biometric sensor to constantly measure the biometric data of the user and to acquire consecutively measured biometric data. According to an embodiment, the processor 120 may cause a biometric sensor to measure the biometric data of the user, based on a specific interruption (e.g., detection of a user request, detection of an entrance into a configured place, or the like) and to acquire consecutively measured biometric data. According to various embodiments, operation 803 and operation 805 may be performed sequentially, in parallel, or inversely.

In operation 807, the processor 120 may match the biometric data with the place and store the matched data. According to an embodiment, the processor 120 may match the consecutively measured biometric data with the place where the consecutive measurement data is measured, and store the matched data. According an embodiment, when the place where the biometric data is measured corresponds to, for example, a specific place (e.g., home, an office, a car, or the like) configured by the user, the processor 120 may update a corresponding place item with the measured data. For example, since there may be biometric data previously measured (or measured at a different time slot) in the corresponding place item, the processor 120 may add currently measured data to the biometric data previously measured, in the corresponding place item, according to a time sequence.

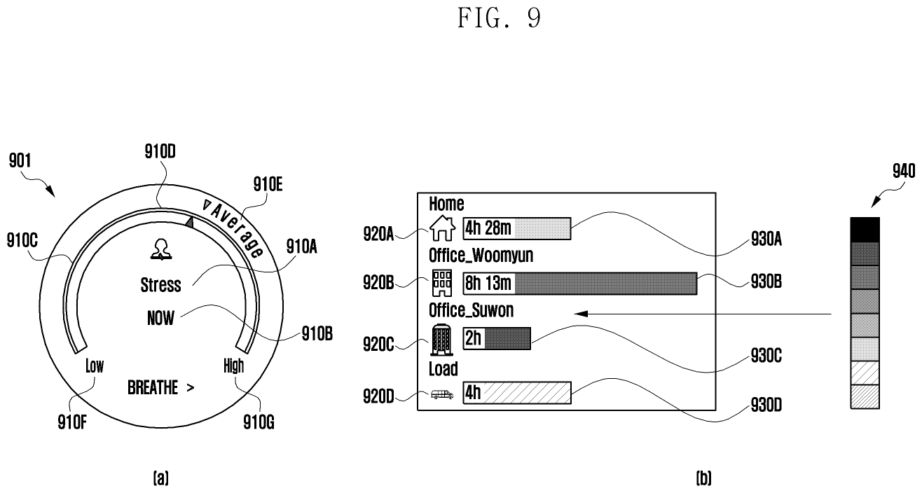

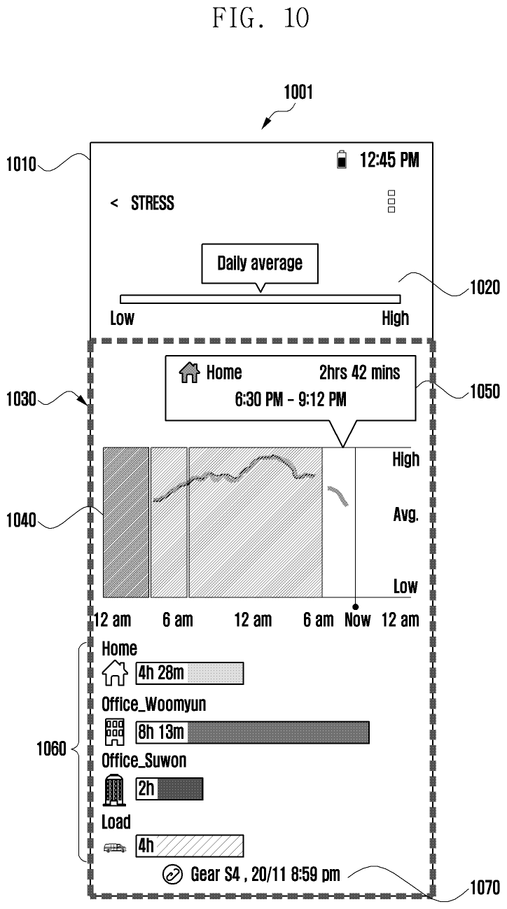

In operation 809, the processor 120 may provide biometric information according to a place. According to an embodiment, when a user input for identifying biometric information is detected, the processor 120 may configure, based on the biometric data, biometric information recognizable by the user, and may display the biometric information through the display device 160. According to an embodiment, when displaying the biometric information, the processor 120 may display related information according to a time sequence, and may display the biometric information by classifying the biometric information according to a duration for each place. According to various embodiments, an example of providing biometric information according to a place is shown in FIGS. 9 and 10.

FIG. 9 illustrates an example of a screen for providing biometric information by an electronic device according to various embodiments.

According to various embodiments, FIG. 9 illustrates an example of providing biometric information (e.g., stress information) by a wearable device, wherein a display device (e.g., the display device 160 of FIG. 1) of the electronic device 101 may have a shape of a wearable device 901, the size of which is relatively small.

Referring to FIG. 9, example A shows an example of a screen on which stress information is displayed through a display device (e.g., the display device 160 of FIG. 1), wherein the stress information is biometric information generated based on biometric data measured by the wearable device 901 (or biometric data acquired by synchronization with another electronic device (e.g., a smartphone) of a user account). According to an embodiment, the displayed stress information of FIG. 9 may be information generated from consecutively measured previous biometric data, or may be information generated from consecutively measured current biometric data. According to an embodiment, for the stress information, various stress values (or stress indices) may be obtained according to the measured biometric data (e.g., a stress state of the user). According to various embodiments, the stress may indicate a symptom caused by a physical or psychological tension. According to an embodiment, the wearable device 901 may measure a change in inter-beat intervals (e.g., heart rate variation (HRV)), may compare the heart rate variation data of the user with that of a group of health people at the user's age, and may provide a stress level of the user.

According to an embodiment, an interface providing stress information in the wearable device 901 may include, for example, an object 910A indicating a type (or an item) of biometric information, an object 910B indicating a measurement time of biometric information, an object 910C indicating a value (or an index) related to biometric information, an object 910D indicating biometric information (e.g., stress information) (or a measurement value) based on measured biometric data, an object 910E indicating an average value (e.g., an average of pieces of data of a group of people at the user's age) relating to biometric information, and an object 910F or 910G indicating a reference so that the user may identify the level (e.g., low or high) of the biometric information of the user. According to an embodiment, the above-described objects may be configured in various ways based at least on text, an icon, an image, a chart, a graphic, or the like, according to a representation scheme (e.g., a numeral value, an image, or text, etc.) of information to be indicated by each of the objects.

Referring to FIG. 9, according to various embodiments, example B shows an example of an interface providing biometric information according to a place. The biometric information is exemplified by stress information in example B.

According to an embodiment, when a user input (or touch) (e.g., an input related to a request to display detailed information) is detected while displaying biometric information relating to the user through the interface (hereinafter, referred to as a “first interface”) as in example A, the wearable device 901 may switch the interface from the first interface to an interface (hereinafter, referred to as a “second interface”) as in example B and display the second interface.

As shown in example B, the second interface may include, for example, a place object 920A, 920B, 920C, or 920D for identifying a place, and a state object 930A, 930B, 930C, or 930D indicating a state of biometric information related to the corresponding place. According to an embodiment, referring to example B, the state object may be provided according to places (e.g., home (e.g., Home), Office 1 (e.g., Office_Woomyun), Office 2 (e.g., Office Suwon), a car (e.g., Car), and the like) registered by the user by using the user account. In example B, four places are exemplified, but information relating to the place may be provided through objects, wherein the number of objects corresponds to the number of places registered by the user.

According to various embodiments, the wearable device 901 may provide biometric information (e.g., stress information) relating to the user by classifying the biometric information according to a place where related biometric data is acquired (or measured). Referring to example B, the wearable device 901 may provide biometric information, for example, a specific (or one) piece of biometric information (e.g., stress information) acquired until now for a day by classifying (or segmenting) the biometric information according to a place. According to an embodiment, the wearable device 901 may divide the biometric information into first biometric information, second biometric information, third biometric information, and fourth biometric information, wherein the first biometric information corresponds to accumulated information related to a first place 920A (e.g., home), the second biometric information corresponds to accumulated information related to a second place 920B (e.g., office 1), the third biometric information corresponds to accumulated information related to a third place 920C (e.g., office 2), and the fourth biometric information corresponds to information related to a fourth place 920D (e.g., a car), and may provide the biometric information by using a state object 930A, 930B, 930C, or 940D corresponding to each piece of biometric information according to a corresponding place.

According to an embodiment, the first biometric information, the second biometric information, the third biometric information, and the fourth biometric information may indicate at least one piece of biometric information (e.g., individual stress information) consecutively acquired or inconsecutively acquired (e.g., acquired at each measurement time slot) at the corresponding place, regardless of a time slot at which related biometric data is acquired (or measured). The wearable device 901 may accumulate (or collect) pieces of biometric information for each place, and obtain an average of the pieces of accumulated biometric information, and provide a state object 930A, 930B, 930C, or 930D corresponding to each place. For example, referring to example B, the wearable device 901 may obtain an average of pieces of biometric information acquired while the user stays at the first place 920A (e.g., home), and may provide a state object 930A relating to the first biometric information.

According to an embodiment, the state objects 930A, 930B, 930C, and 930D may be provided in different colors according to an obtained value (e.g., a stress index) of biometric information, and may be provided in the same color or in different colors for each place according to a value of biometric information for each place. For example, as shown by reference numeral 940, the state objects 930A, 930B, 930C, and 930D may be represented in eight stages of color according to the stress index. An example thereof is shown by reference numeral 940. According to an embodiment, the stress index is divided into a first state (e.g., best) to an eighth state (e.g., worst), and the first state to the eighth state may be represented in color, corresponding to a first color (e.g., green) to an eighth color (e.g., orange), respectively.

According to an embodiment, the state objects 930A, 930B, 930C, and 930D may be provided including time information relating to a time for which the user has stayed at the corresponding place. For example, referring to example B, the user has stayed at the first place 920A (e.g., home) for 4 hours and 28 minutes (4 h 28 m), the user has stayed at the second place 920B (e.g., office 1) for 8 hours and 13 minutes (8 h 13 m), the user has stayed at the third place 920C (e.g., office 2) for 2 hours (2 h), and the user has stayed at the fourth place 920D (e.g., a car) for 4 hours (4 h). According to an embodiment, the time for which the user has stayed at each place may be a time for which the user has consecutively stayed at the corresponding place, or may be a time obtained by combining each time for which the user has inconsecutively stayed at the corresponding place.

FIG. 10 illustrates an example of a screen for providing biometric information by an electronic device according to various embodiments.

According to various embodiments, FIG. 10 illustrates an example of providing biometric information by a smartphone, wherein a display device (e.g., the display device 160 of FIG. 1) of the electronic device 101 may have a shape of a smartphone (or a tablet PC) 1001, the size of which is relatively large.

Referring to FIG. 10, FIG. 10 may show an example of a screen on which stress information is displayed through a display device (e.g., the display device 160 of FIG. 1), wherein the stress information is biometric information generated based on biometric data measured by the smartphone 1001 (or biometric data acquired by synchronization with another electronic device (e.g., a wearable device) of a user account).

According to an embodiment, an interface providing stress information by the smartphone 1001 may include, for example, a region 1010 indicating a type (or an item) of biometric information, a region 1020 indicating information relating to a measurement time (e.g., Thur, 20 November) of provided biometric information and an average (e.g., a daily average) of pieces of biometric information at the corresponding measurement time, and a region 1030 indicating detailed information related to biometric information. According to an embodiment, although it is not shown in FIG. 10, the interface may include more regions than the above-described regions. For example, in response to a user input (e.g., a scroll input), a screen may be scrolled to be displayed by the smartphone 1001, whereby new information may be indicated according to the scrolling.

According to an embodiment, the region 1030 (or an interface) indicating detailed information related to biometric information may include a chart region 1040 for providing accumulated biometric information (e.g., accumulated stress information) measured for a measurement time (e.g., for a day) in a form of a chart (or a graph), a duration information region 1050 for providing time information and place information related to a region (or a duration) selected (or touched) by a user in the chart region 1040, and a place information region 1060 for providing accumulated biometric information measured for a measurement time by classifying the accumulated biometric information according to a place. According to an embodiment, the region 1030 may include a source region 1070 for providing information (e.g., Gear S4, 20/11 8:59 pm) relating to a source of the biometric information. According to an embodiment, the information relating to the source may include, for example, information relating to a device (e.g., a wearable device) by which biometric information is acquired, and time information relating to a time (or a synchronization time, etc.) at which biometric information is acquired from the corresponding device.

According to an embodiment, the place information region 1060 may correspond to the description on the second interface being referred to in example B of FIG. 9, and detailed description thereof is omitted.

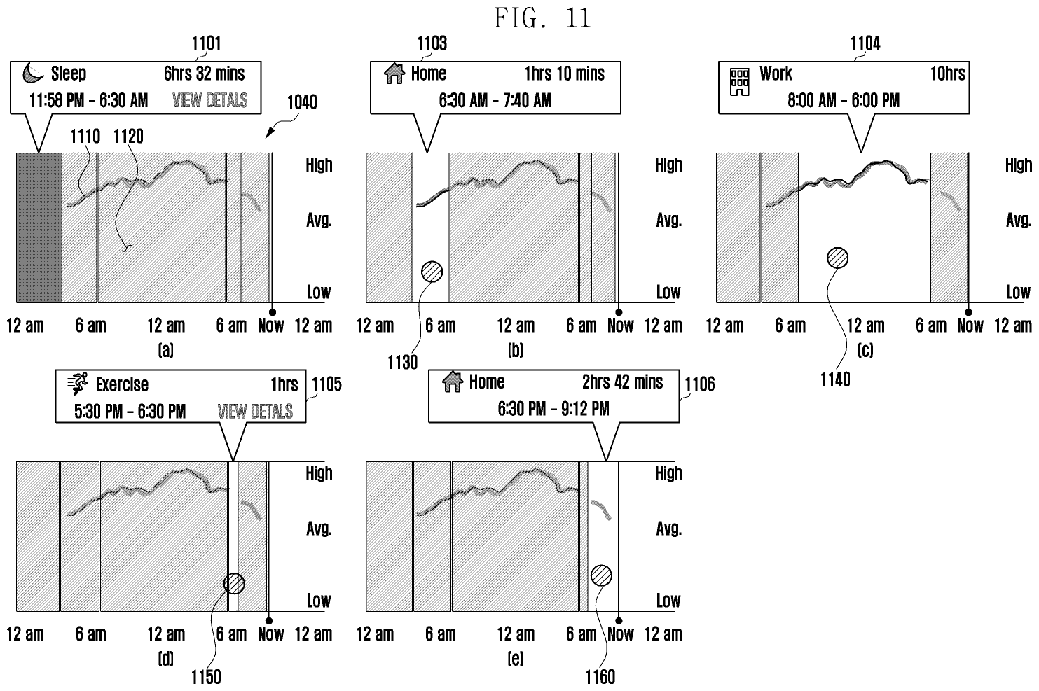

According to an embodiment, the smartphone 1001 may provide the duration information region 1050 in response to a user input (or selection) from the chart region 1040. According to various embodiments, when the user input is detected from the chart region 1040, the smartphone 1001 may determine a place corresponding to the user input, and may provide biometric information related to the determined place in the duration information region 1050. The detailed example thereof will be described in FIG. 11.

FIG. 11 illustrates an example of a screen for providing biometric information by an electronic device according to various embodiments.

Referring to FIG. 11, screen example A to screen example E in FIG. 11 may indicate the chart region 1040 and the duration information region 1050 described in the part described with reference to FIG. 10. According to an embodiment, the electronic device 101 may classify a duration according to a place related to the user, and display only biometric information at the corresponding place by highlighting the biometric information, thereby allowing the user to more intuitively and effectively recognize the biometric information at the corresponding place.

Referring to screen example A, the chart region 1040 may represent accumulated biometric information (e.g., accumulated stress information) measured for a measurement time (e.g., for a day) in a form of a graph 1110. In the chart region 1040, the X-axis indicates a time (e.g., from 12 AM until now), and the Y-axis indicates a stress index (or a stress measurement value). According to an embodiment, as shown in screen example A, when displaying the chart region 1040, the electronic device 101 may display the chart region 1040 by shading the region. For example, the electronic device 101 may display the chart region 1040 by highlighting the graph 1110 in the chart region 1040, and applying the brightness and the color to the entire region. According to various embodiments, for example, the graph 1110 may be provided based on the above-described eight stages of color. According to various embodiments, the displaying operation by the shading the chart region 1040 may be selectively performed according to the configuration of the electronic device 101.

Screen example A may indicate an example of selecting (or activating) a sleep duration corresponding a sleep state of the user during the measurement time, and accordingly, providing information related to the sleep duration in the duration information region 1101. According to an embodiment, the user may have no stress or stress information may be meaningless when the user is in the sleep state, and thus no stress information may be provided in the sleep duration. Accordingly, the duration information region 1101 may indicate that the user is in the sleep state in the corresponding duration, and may provide information including time information (e.g., a sleep start time, a sleep end time, total sleeping hours, etc.) related to the sleep state. In addition, the duration information region 1101 may further include an item (e.g., VIEW DETAILS) by which detailed information (e.g., the level of tossing and turning in sleep, sleep efficiency, calorie consumption, etc.) relating to various sleep states of the user may be identified (or through which access to detailed information is available). According to an embodiment, the detailed information relating to the sleep state may be provided as numeric information for each item such as sleep efficiency, actual sleeping hours, no tossing and turning hours, hours for which the user has tossed and turned less, hours for which the user tossed and turned a lot, calorie consumption, etc.

Referring to screen example B to screen example E, screen example B to screen example E may show examples of providing biometric information by classifying the biometric information according to a place when the user selects (or touches) a certain region from the chart region 1040. According to an embodiment, the electronic device 101 may determine, in response to the selection of a certain region by the user from the chart region 1040, a place (or a place duration) corresponding to the selected region. According to an embodiment, the electronic device 101 may intuitively indicate, by highlighting a region corresponding to the determined place (or the determined place duration) in the chart region 1040, a graph of biometric information at the corresponding place, and may provide detailed information related to the biometric information at the corresponding place through duration information regions 1103, 1104, 1105, and 1106.

In various embodiments, the highlighting of the region corresponding to the place (or the place duration) may be, for example, a scheme of highlighting only a duration (e.g., a partial region of the chart region 1040) corresponding to the selected place (or the selected place duration), in the shaded region of the chart region 1040 (e.g., the entire region of the chart region 1040). For example, the electronic device 101 may extract a duration (e.g., a duration (or a time duration) including consecutive measurement data at a selected place) corresponding to the place (e.g., home, an office, a car, or a performance venue, etc.) selected from the chart region 1040, determine a range (e.g., a start point to an end point) corresponding to the extracted duration, and highlight the determined range. According to an embodiment, the highlighting, for example, corresponds to highlighting (or intuitively providing) biometric information at a place corresponding to a user selection in the chart area 1040, and may include various schemes such as making a part to be highlighted flicker, marking a bold line on a part to be highlighted, increasing the level of contrast of a part to be highlighted, applying reverse video (e.g., reversing a black-and-white part of a screen) to a part to be highlighted, or coloring a part to be highlighted, etc.

According to an embodiment, as shown in screen example B, screen example B may show an example of selecting (e.g., touching) a region 1130 from the chart region 1040 by the user. The electronic device 101 may determine a place (e.g., home) corresponding to the selected region 1130, and may determine measurement data (e.g., consecutive measurement data) during the user's stay at the determined place. The electronic device 101 may determine, based on the measurement data, a duration corresponding to the determined place, and may highlight the determined duration. According to an embodiment, the electronic device 101 may provide duration information of the selected region 1130 to the user through the duration information region 1130. For example, the electronic device 101 may display information relating to a place (e.g., Home) corresponding to the selected region 1103, and first information (e.g., time information, 6:30 AM-7:40 AM) and second information (e.g., information on a total sum of hours for which the user has stayed, 1 hrs 10 mins) relating to a time for which the user has stayed at the corresponding place (or a measurement time at the corresponding place).

According to an embodiment, as shown in screen example C, screen example C may show an example of selecting (e.g., touching) a region 1140 from the chart region 1040 by the user. The electronic device 101 may determine a place (e.g., an office) corresponding to the selected region 1140, and may determine measurement data (e.g., consecutive measurement data) during the user's stay at the determined place. The electronic device 101 may determine, based on the measurement data, a duration corresponding the determined place, and may highlight the determined duration. According to an embodiment, the electronic device 101 may provide duration information of the selected region 1140 to the user through the duration information region 1104. For example, the electronic device 101 may display information relating to a place (e.g. Work) corresponding to the selected region 1140, and first information (e.g., 8:00 AM-6:00 PM) and second information (e.g., 10 hrs) relating to a time for which the user has stayed at the corresponding place (or a measurement time at the corresponding place).

According to an embodiment, as shown in screen example D, screen example D may show an example of selecting (e.g., touching) a region 1150 from the chart region 1040 by the user. The electronic device 101 may determine a place (e.g., a workout place) corresponding to the selected region 1150, and may determine measurement data (e.g., consecutive measurement data) during the user's stay at the determined place. The electronic device 101 may determine, based on the measurement data, a duration corresponding to the determined place, and may highlight the determined duration. According to an embodiment, the electronic device 101 may provide duration information relating to the selected region 1150 to the user through the duration information region 1105. For example, the electronic device 101 may display information (e.g., Exercise) relating to a place corresponding to the selected region 1150, and first information (e.g., 5:30 PM-6:30 PM) and second information (e.g., 1 hrs) relating to a time for which the user has stayed at the corresponding place (or a measurement time at the corresponding place).

According to an embodiment, as shown in screen example C and screen example D, some durations for a specific place of the chart region 1040 may overlap with each other. For example, a part of the duration in screen example C may be classified as a duration at another place. For example, while staying at office, the user may workout at a workout space of a specific place (e.g., on a different floor in the same building) in the office. In this case, the place where the user stays may be an office, and information on a workout place (or duration) may be provided separately from the place of office.

According to an embodiment, as shown in screen example E, screen example E may show an example of selecting (e.g., touching) a region 1160 from the chart region 1040 by the user. The electronic device 101 may determine a place (e.g., home) corresponding to the selected region 1160, and may determine measurement data (e.g., consecutive measurement data) during the user's stay at the determined place. The electronic device 101 may determine, based on the measurement data, a duration corresponding to the determined place, and may highlight the determined duration. According to an embodiment, the electronic device 101 may provide duration information relating to the selected region 1160 to the user through the duration information region 1106. For example, the electronic device 101 may display information (e.g., Home) relating to a place corresponding to the selected region 1160, and first information (e.g., 6:30 PM-9:12 PM) and second information (e.g., 2 hrs 42 mins) relating to a time for which the user has stayed at the corresponding place (or a measurement time at the corresponding place).

According to an embodiment, screen example E may indicate a state in which the user is staying at a place corresponding to the selected region 1160 until now. The electronic device 101 may display the duration from a time at which the user starts staying at the corresponding place until now (e.g., NOW) by highlighting the duration.

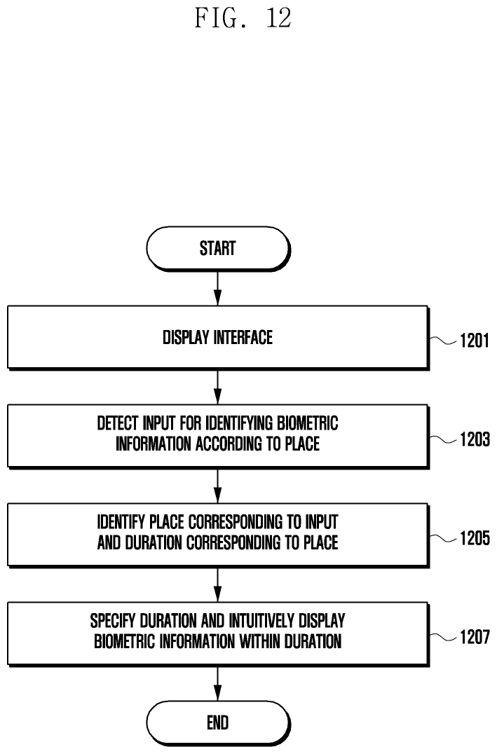

FIG. 12 is a flowchart illustrating an operation method of an electronic device according to various embodiments.

Referring to FIG. 12, in operation 1201, the processor 120 of the electronic device 101 may display an interface. According to an embodiment, the processor 120 may display an interface for providing the biometric information as illustrated in FIGS. 9 to 11, through the display device 160 upon a user request.

In operation 1203, the processor 120 may detect an input for identifying biometric information according to a place through an interface. According to an embodiment, as shown in FIG. 11, the processor 120 may detect a user input of selecting (e.g., touching) a certain region from the chart region 1040 by the user.

In operation 1205, the processor 120 may identify a place corresponding to the input, and a duration corresponding to the place. According to an embodiment, the processor 120 may determine, in response to the selection of a certain region from the chart region 1040 by the user, a place corresponding to the selected region and a place duration (or range) including the corresponding place.

In operation 1207, the processor 120 may specify a duration and display biometric information within the duration by intuitively emphasizing (e.g., highlighting) the biometric information. According to an embodiment, as illustrated in FIG. 11, the processor 120 may intuitively indicate, by highlighting a region corresponding to a determined place (or a place duration), among the chart region 1040, a graph of biometric information at the corresponding place, and may provide detailed information related to the biometric information at the corresponding place through a duration information region.

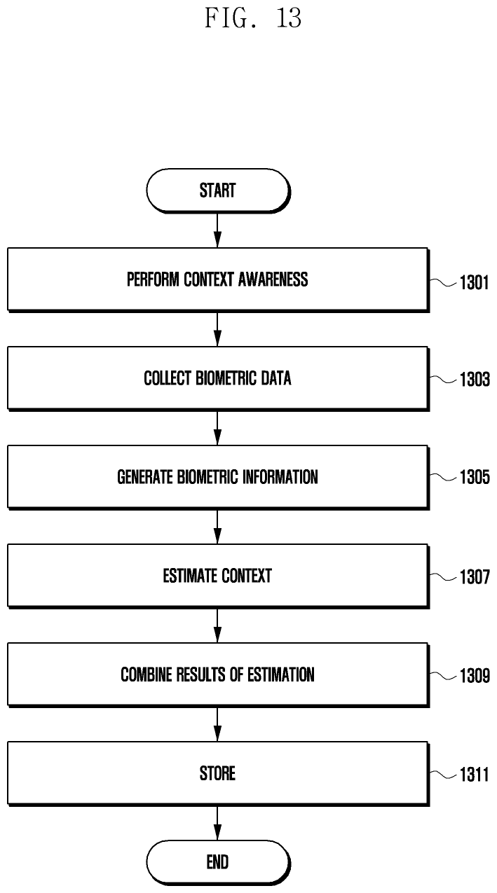

FIG. 13 is a flowchart illustrating an operation method of an electronic device according to various embodiments.

Referring to FIG. 13, in operation 1301, the processor 120 of the electronic device 101 may perform context awareness. According to an embodiment, the processor 120 may recognize (or monitor) various contexts related to the electronic device 101 (or a user) by using context awareness technology. According to an embodiment, the processor 120 may determine a context by analyzing data (or information) input from various sensors (e.g., the sensor module 176 of FIG. 1). According to an embodiment, the processor 120 may recognize various contexts such as an application executed by the electronic device 101, an event related to a state (e.g., emotion) of the user, a place where the user is located, or a stay time.

In operation 1303, the processor 120 may collect biometric data relating to the user. According to an embodiment, the processor 120 may cause a biometric sensor to constantly measure the biometric data of the user and to acquire consecutively measured biometric data. According to an embodiment, the processor 120 may cause a biometric sensor to measure, based on a specific interruption (e.g., detection of a user request, detection of an entrance into a configured place, or the like), the biometric data of the user and to acquire consecutively measured biometric data. According to various embodiments, operation 1301 and operation 1303 may be performed sequentially, in parallel, or inversely.

In operation 1305, the processor 120 may generate biometric information. According to an embodiment, the processor 120 may generate at least one piece of biometric information satisfying a condition (e.g., a measurement time or an amount of measurement data) required for the biometric information, based on the collected biometric data. According to an embodiment, the processor 120 may generate stress information by using consecutive biometric data for a predetermined time. According to another embodiment, the processor 120 may generate emotion information by using the consecutive biometric data for a predetermined time.

In operation 1307, the processor 120 may perform context estimation. According to an embodiment, the processor 120 may estimate the result of the context awareness, and also estimate a user state according to the biometric information. For example, the processor 120 may estimate a place where the user is currently located, as a result of the context awareness. For example, the processor 120 may estimate an application executed by the electronic device 101, as a result of the context awareness. For example, the processor 120 may estimate contents used (or performed) through the application, as a result of the context awareness. For example, the processor 120 may estimate a stress level (or an emotional state) of the user, such as high stress, moderate stress, low stress, etc., based on the biometric information. According to various embodiments, the processor 120 may determine whether the stress level (or the emotion) of the user is good or bad (e.g., a relative level of good or bad determined based on a specific condition).

According to various embodiments, the processor 120 may determine whether a stress level of a user of a current application is good or bad. According to various embodiments, the processor 120 may estimate a context in which the user gets stress and the level of stress that the user gets (or the type of emotion that the user has), based on the context awareness and the biometric information. For example, the processor 120 may estimate a change in states according to contexts of the user, and may classify the user state according to the context. An example thereof is described with reference to [Table 2] below.

In operation 1309, the processor 120 may combine the results of the estimation. According to an embodiment, the processor 120 may generate at least one result according to the context awareness, as context data (or combined data). For example, the processor 120 may match place information of the current user with the stress information of the current user. For example, when an application is executed, the processor 120 may match application information of the application with stress information. For example, the processor 120 may match place information of the current user, application information, and stress information. According to an embodiment, the processor 120 may classify a state according to a context of the user based at least on the place information, the application information, and the stress information, and may match the place information, the application information, and the stress information.

In operation 1311, the processor 120 may store context data (or information). According to various embodiments, as shown in [Table 2] below, the context data (or information) may be matched by analyzing a context relating to the electronic device 101 (or a user) and biometric information to be measured, and extracting and classifying data affecting (e.g., positively affecting, negatively affecting, etc.) stress level (or emotion) of the user.

TABLE 2

| No. | Usage data | Extracted data | Scheme |
|---|---|---|---|
| 1 | Body sensing | Who makes a user's heart beat | Identify an HR and a stress value |
| | data: HR, Stress | faster? | measured from a call start time |
| | Phone usage: Call | : Contact1, Contact2, | to a call end time, and extract a |
| | log | Contact3 . . . | person who made the HR of the |
| | | | user increase beyond an average |
| | | | HR of the user |
| 2 | | Who makes a user feel | Identify an HR and a stress value |
| | | comfortable (stable)? | measured from a call start time |
| | | : Contact4, Contact5, | to a call end time, and extract a |
| | | Contact6 . . . | person who made the HR and the |
| | | | stress value of the user decrease |
| | | | under an average HR and stress |
| | | | value of the user |
| 3 | | Who makes a user get | Identify an HR and a stress value |
| | | exhausted? | measured from a call start time |
| | | : Contact7, Contact8, | to a call end time, and extract a |
| | | Contact9 . . . | person who made the HR and the |
| | | | stress value of the user increase |
| | | | beyond an average HR and stress |
| | | | value of the user |
| 4 | Body sensing | Which contents such as music | Identify an HR and a stress value |
| | data: HR, Stress | or video make a user's heart | measured from a playback start |
| | Phone usage: | beat faster? | time to a playback end time of |
| | Music, Video, | : Music1, Music2, Music3 . . . | contents, and extract contents |
| | Internet | : Video1, Video2, Video3 . . . | which made the HR of the user |
| | | | increase beyond an average HR |
| | | | of the user |
| 5 | | Which contents such as music | Identify an HR and a stress value |
| | | or video make a user feel | measured from a playback start |
| | | comfortable? | time to a playback end time of |
| | | : Music4, Music5, Music6 . . . | contents, and extract contents |
| | | : Video4, Video5, Video6 . . . | which made the HR and the |
| | | | stress value of the user decrease |
| | | | under an average HR and stress |
| | | | value of the user |
| 6 | | Which contents such as music | Identify an HR and a stress value |
| | | or video make a user feel | measured from a playback start |
| | | uncomfortable? | time to a playback end time of |
| | | : Music1, Music2, Music3 . . . | contents, and extract contents |
| | | : Video1, Video2, Video3 . . . | which made the HR and the |
| | | | stress value of the user increase |
| | | | beyond an average HR and stress |
| | | | value of the user |
| 7 | Body sensing | Which app makes a user be | Identify an HR and a stress value |
| | data: HR, Stress | pleasant? | measured from an app usage start |
| | Phone usage: All | : App1, App2, App3 . . . | time to an app usage end time, |
| | apps | | and extract an app which made a |
| | | | positive stress value increase |
| 8 | | Which app makes a user feel | Identify an HR and a stress value |
| | | comfortable? | measured from an app usage start |
| | | : App4, App5, App6 . . . | time to an app usage end time, |
| | | | and extract an app which made |
| | | | the HR and stress value more |
| | | | stable |
| 9 | | Which app makes a user feel | Identify an HR and a stress value |
| | | uncomfortable? | measured from an app usage start |
| | | : App7, App8, App9 . . . | time to an app usage end time, |
| | | | and extract an app which made a |
| | | | negative stress value increase |
| 10 | Body sensing | Which event made a user's | Identify an HR and stress value |
| | data: HR, Stress | heart beat faster? | measured during an event hour |
| | Phone usage: | : Birthday party, meeting | registered to Calendar, and |
| | Calendar | OOO . . . | extract an event which made the |
| | | | HR of the user increase beyond |
| | | | an average HR of the user and an |
| | | | event in which positive stress is |
| | | | measured |
| 11 | | In which event did a user feel | Identify an HR and stress value |
| | | relaxed? | measured during an event hour |
| | | : participation in corporate | registered to Calendar, and |
| | | retreat, club BB . . . | extract an event which made the |
| | | | HR of the user closer to the |
| | | | resting HR and an event in which |
| | | | calm stress is measured |
| 12 | | In which event did a user feel | Identify an HR and stress value |
| | | uncomfortable | measured during an event hour |
| | | : Meeting □□□, club AA, | registered to Calendar, and |
| | | exam . . . | extract an event which made the |
| | | | HR of the user increase far |
| | | | beyond the resting HR and an |
| | | | event in which data shows an |
| | | | increase in a value of negative |
| | | | stress |

As shown in [Table 2], according to various embodiments, an example of utilizing extracted data (e.g., context awareness data, measurement biometric data, etc.) is described in [Table 2]. For example, referring to item 1, item 2, and item 3 in [Table 2], an example of combining biometric information (e.g., HR or stress) acquired based on the biometric data with a usage log (e.g., a Call log) of the electronic device 101 so as to estimate and configure a person (user) (e.g., Contact 1, Contact 2, Contact 3, etc.) who made a user be in a first state (e.g., a state in which the user is pleasantly excited) (or who made the user's heart beat faster), a person (user) (Contact 4, Contact 5, Contact 6, etc.) who made a user be in a second state (e.g., a state in which the user feels comfortable (or stable)), and a person (user) (e.g., Contact 7, Contact 8, Contact 9, etc.) who made a user be in a third state (e.g., a state in which the user feels exhausted) is described. According to an embodiment, in the case of item 1, for example, an HR and a stress value measured from a call start time to a call end time may be identified so that a counterpart user who made the HR of a user increase beyond an average HR of the user is estimated. According to an embodiment, in the case of item 2, an HR and a stress value measured from a call start time to a call end time may be identified so that a counterpart user who made the HR and the stress value of a user decrease under an average HR and stress value of the user is estimated. In the case of item 3, an HR and a stress value measured from a call start time to a call end time may be identified so that a counterpart user who made the HR and the stress value of a user increase beyond an average HR and stress value of the user is estimated.

According to various embodiments, the processor 120 may provide various types of feedback to a user, based on the information configured as shown in [Table 1]. According to an embodiment, the processor 120 may analyze consecutive stress measurement data and an amount of changes in stress averages, and may recommend a person (e.g., another user) positively affecting the user, an application (or an app), contents, an event, or the like when the user needs to calm his or her mind. According to various embodiments, the processor 120 may provide, to the user, an insight (or guidance or a tip) for helping a user know how to handle contexts in which negative stress occurs.

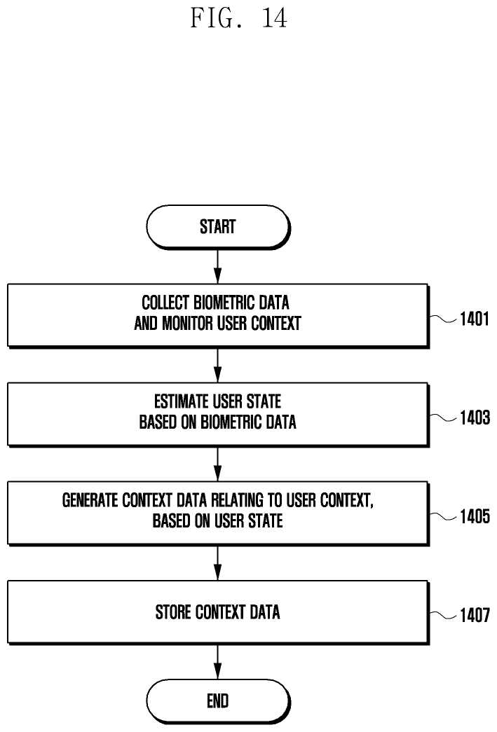

FIG. 14 is a flowchart illustrating an operation method of an electronic device according to various embodiments.

As illustrated in FIG. 14, an example of an operation of generating context data as shown in above-described [Table 2] is described in FIG. 14.

Referring to FIG. 14, in operation 1401, the processor 120 of the electronic device 101 may collect biometric data and monitor a user context.

In operation 1403, the processor 120 may estimate a user state based on the biometric data. According to an embodiment, the processor 120 may estimate whether the user is in a pleasantly excited state, an unpleasantly excited state, a sad state, an angry state, or the like. According to an embodiment, the processor 120 may estimate a user state based at least on at least one piece of biometric information (e.g., stress, an FIR, oxygen saturation, emotion, etc.) which can be acquired from the biometric data. According to an embodiment, referring to [Table 2], when the HR of the user increases beyond an average HR while the user is on the phone or is listening to music, the processor 120 may estimate that the user is in a pleasantly excited state.

In operation 1405, the processor 120 may generate context data relating to the user context based on the user state. According to an embodiment, the processor 120 may generate context data by matching, with a person (user) who makes a user's heart beat faster, a counterpart user who is talking on the phone with the user, and also matching a related context (e.g., usage data (e.g., an HR, stress, or a call log in [Table 2])). According to an embodiment, the processor 120 may generate context data by matching music (or contents), which the user is listening to (or enjoying), with contents which makes the user's heart beat faster, and also matching a related context (e.g., usage data (e.g., an HR, stress, or music in [Table 2])).

In operation 1407, the processor 120 may store the context data. According to an embodiment, the processor 120 may store the context data as shown in [Table 2], in the memory 130 of the electronic device 101. The context data as shown in [Table 2] may be updated or deleted, or a new item may be added to the context data, according to a user state.

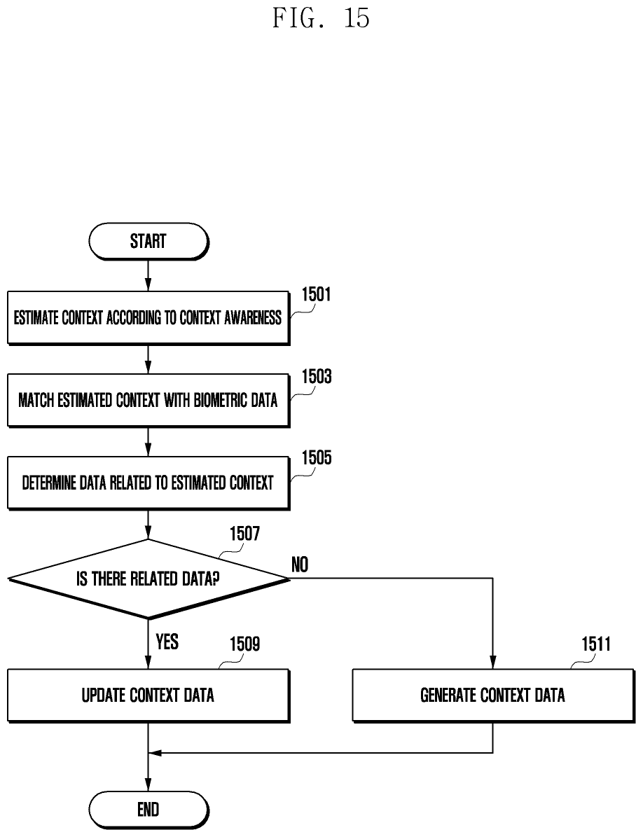

FIG. 15 is a flowchart illustrating an operation method of an electronic device according to various embodiments.

Referring to FIG. 15, in operation 1501, the processor 120 of the electronic device 101 may estimate a context according to context awareness. According to an embodiment, the processor 120 may recognize (or monitor) various contexts related to the electronic device 101 (or a user). According to an embodiment, the processor 120 may determine a context by analyzing data (or information) input from various sensors (e.g., the sensor module 176 of FIG. 1). According to an embodiment, the processor 120 may recognize various contexts such as an application executed in the electronic device 101, an event related to a user state (e.g., emotion), a place where the user is located, a stay time, or the like. According to an embodiment, an operation of acquiring (collecting) biometric data by the processor 120 by using a biometric sensor may be further included, in parallel to, or sequential to operation 1501.

In operation 1503, the processor 120 may match the estimated context with the biometric data.

In operation 1505, the processor 120 may determine data (e.g., the extracted data in [Table 2]) related to the estimated context. According to an embodiment, the processor 120 may identify, from the context data as shown in [Table 2] above, an item corresponding to the estimated context and extracted data related to the corresponding item.

In operation 1507, the processor 120 may determine whether data related to the estimated context exists.

When data related to the estimated context exists in operation 1507 (if “YES” in operation 1507), the processor may update the context data with context data related the estimated context in operation 1509. According to an embodiment, the processor 120 may update context data with extracted data. For example, referring to [Table 2], when the estimated context corresponds to item 1, the processor 120 may update context data by adding a subject user (e.g., Contact X) to the extracted data in item 1. In another example, referring to [Table 2], when a subject user (e.g., Contact 3) is included in the extracted data, but no estimated context corresponds to item 1, the processor 120 may update context data by deleting the subject user (e.g., Contact 3) from the extracted data of item 1.

When data related to the estimated context does not exist in operation 1507 (if “NO” in operation 1507), the processor 120 may generate related context data in operation 1511. According to an embodiment, the processor 120 may generate context data including a new item and related extracted data.

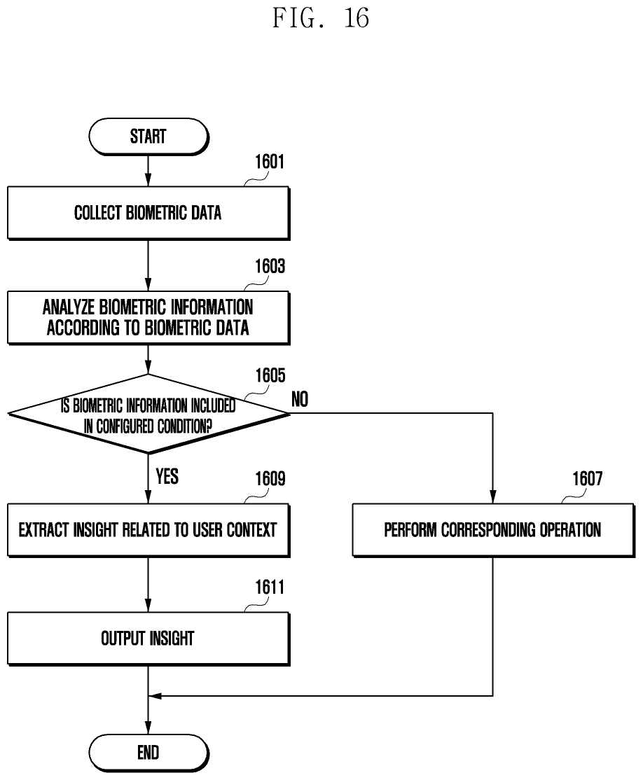

FIG. 16 is a flowchart illustrating an operation method of an electronic device according to various embodiments.

Referring to FIG. 16, in operation 1601, the processor 120 of the electronic device 101 may collect biometric information relating to a user. According to an embodiment, the processor 120 may constantly collect biometric data of the user in a state in which the electronic device is worn on the user's body. According to an embodiment, the processor 120 may collect biometric data constantly (or in a push or pull scheme) from another electronic device worn on the user's body.

In operation 1603, the processor 120 may analyze biometric information according to the biometric data. According to an embodiment, the processor 120 may acquire biometric information based on the collected biometric data, and may determine a specific value (e.g., a stress index, etc.) according to the biometric information by analyzing the acquired biometric information. For example, the processor 120 may compare the biometric information with reference information (or user's average biometric information (e.g., information on an average of the user's stress levels), or an average of pieces of data of a group of healthy people at the user's age, etc.) preconfigured to determine the level of the biometric information, so as to determine the level of the biometric information (e.g., low or high stress index, etc.).

In operation 1605, the processor 120 may determine whether the biometric information is included in a configured condition. According to an embodiment, the configured condition may indicate a reference for determining whether to output an insight according to the user's biometric information. According to an embodiment, the processor 120 may determine whether the biometric information has a value lower or higher than the configured condition. For example, the processor 120 may determine that the user's stress level is high when the biometric information (e.g., a stress index) is included in the configured condition (e.g., a condition equal to or greater than a value of the reference information), and may determine that the user's stress level is low when the biometric information is not included in the configured condition. According to various embodiments, the processor 120 may determine whether to output a related insight according to whether the biometric information is included in the configured condition.

When the biometric information is not included in the configured condition in operation 1605 (e.g., if “NO” in operation 1605), the processor 120 may perform the corresponding operation in operation 1607. According to an embodiment, the processor 120 may output the biometric information to the user, or may internally manage the biometric information (e.g., accumulate the biometric information), without outputting the biometric information. According to an embodiment, the processor 120 may manage the biometric information by matching the biometric information with a current place.

When the biometric information is included in the configured condition in operation 1605 (if “YES” in operation 1605), the processor 120 may extract an insight (e.g., guidance or tips, etc.) related to a user state in operation 1609. According to an embodiment, the processor 120 may extract a related insight based at least on the biometric information and context information. For example, the processor 120 may perform context awareness when collecting the biometric information, and may extract an insight corresponding to the level (e.g., a stress index) of a value of the biometric information and context information (e.g., place information) according to the context awareness. According to an embodiment, when there is no related insight, the processor 120 may further include an operation of generating an insight corresponding to the context information and the biometric information.

In operation 1611, the processor 120 may output the insight. According to an embodiment, the processor 120 may display the extracted insight as visual information through the display device 160. According to an embodiment, the processor 120 may output audio data related to the extracted insight as auditory information through the sound output device 155 (e.g., a speaker). According to various embodiments, the processor 120 may output both the visual information and the auditory information, and may also output tactile information (e.g., vibration for alarming the output of the insight).

In FIG. 16, an example of providing an insight in real time based on a configured condition whenever the electronic device 101 collects biometric information of the user is described, however, various embodiments are not limited thereto. According to various embodiments, the electronic device 101 may provide an insight based on biometric information accumulated according to a configured period (e.g., a time unit, a day unit, a week unit, a specific time (e.g., breakfast, lunch, dinner, etc.) unit, or a week unit, etc.). According to various embodiments, the electronic device 101 may provide a related insight when it is detected that biometric information (or accumulated biometric information) is included in a configured condition (e.g., when measured stress information or accumulated stress information has a value higher than a configured stress index). According to various embodiments, the electronic device 101 may determine, based on the existing history, an entrance into a place in which the user's stress was high, and may provide an insight when the user enters the place.

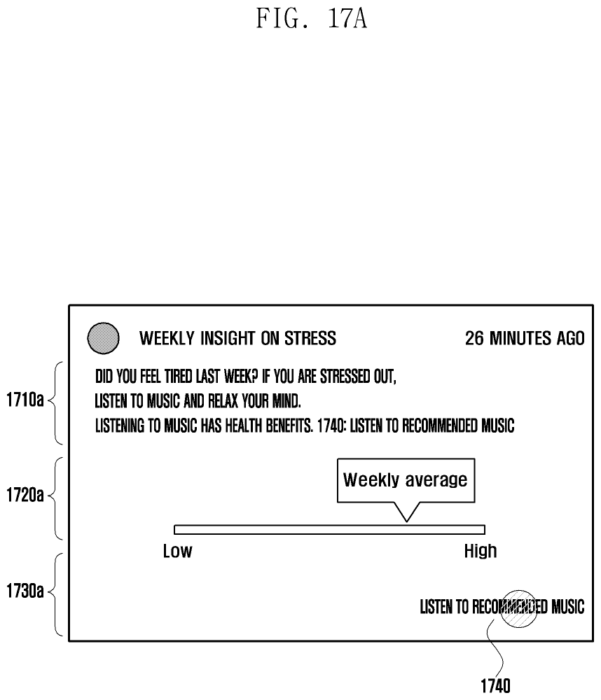

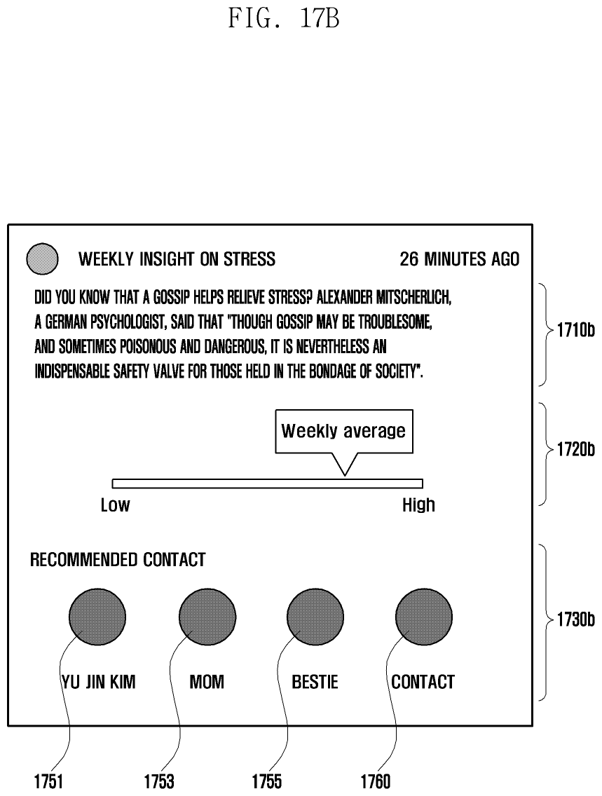

FIGS. 17A and 17B illustrate examples of screens for providing an insight by an electronic device according to various embodiments.

Referring to FIGS. 17A and 17B, according to various embodiments, FIGS. 17A and 17B may show examples of providing a related insight (or an insight card) to a user, based on biometric information accumulated for a week. Various embodiments are not limited thereto, and based on related biometric information, an insight may be provided to the user in real time, or according to a time unit or a place unit, etc.

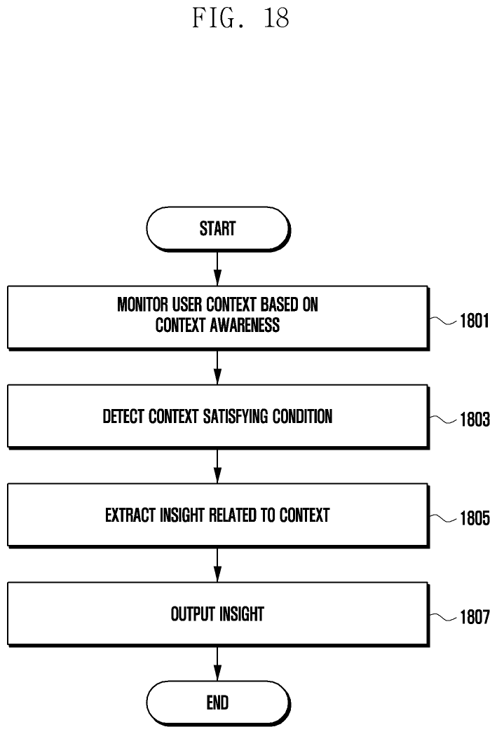

As shown in FIGS. 17A and 17B, FIGS. 17A and 18B may show examples of screens for providing, for example, a weekly insight on stress.

According to various embodiments, an insight (or an insight card) may include a guidance region 1710A or 1710B, a notification region 1720A or 1720B, and a recommendation region 1730A or 1730B.

According to an embodiment, the guidance region 1710A or 1710B may indicate a region in which information (or a phrase) guiding through stress of the user is provided. According to an embodiment, the guidance region 1710A or 1710B may be provided including the contents (or guidance or tips) to be informed of to the user and the purpose thereof. According to an embodiment, a guidance phrase appropriate for a user context may be selected from among various guidance phrases which may be used in the user's various stressful contexts, and the guide region 1710A or 1710B may be provided including the selected guidance phrase. According to an embodiment, the electronic device 101 may search for a required (or appropriate) guidance phrase according to a user context (or a stress level of the user), and may display the found guidance phrase on the guidance region 1710A or 1710B. Alternatively, the electronic device 101 may randomly select a guidance phrase and display the selected guidance phrase on the guidance region 1710A or 1710B. According to an embodiment, the electronic device 101 may provide the guidance phrase by adding required contents to the basic guidance phrase and amending the basic guidance. According to various embodiment, in addition to allowing the user to simply identify information through the guidance of the guidance region 1710A or 1710B, the electronic device 101 may provide a chance for the user who receives the corresponding information to select the corresponding details and make a decision, so as to induce a related action. According to an embodiment, an image (or an emoticon, an icon, etc.) may be attached to the guidance phrase in the guidance region 1710A or 1710B according to a context, thereby facilitating user understanding.

According to an embodiment, the notification region 1720A or 1720B may indicate a region in which biometric information relating to a user is intuitively provided. According to an embodiment, the notification region 1720A or 1720B may be provided including a color-based graph (or a chart) indicating a range of biometric information (or accumulated biometric information (e.g., accumulated stress information)), and a marker (or an indicator (e.g., a speech bubble or a speech balloon, an arrow, an emoticon, etc.)) in a location corresponding to the user's biometric information (or average biometric information) in the graph. Based at least on the graph or the marker of the notification region 1720A or 1720B, the user may intuitively recognize or identify the user's biometric information (or stress information).

According to an embodiment, the recommendation region 1730A or 1730B may indicate a region which induces an action related to the user's stress relief and provides a function object related to the user's stress relief. According to an embodiment, in the recommendation region 1730A or 1730B, a function object related to the guidance phrase provided (or displayed) through the guidance region 1710A or 1710B may be provided.

For example, an example is described in FIG. 17A, wherein guidance of the guidance region 1710A relates to music listening, and a function object 1740 related to music playback is provided in response thereto. For example, an example of providing the function object 1740 for inducing music playback beneficial to the user, according to the guidance of the guidance region 1710A by the electronic device 101 is described. As shown in FIG. 17A, the electronic device 101 may provide, based on the guidance phrase of the guidance region 1710A, a function object 1740 for inducing playback of music which made stress and an HR of the user stable, or to be closer to a stable state, at the time of the user's usual listening. According to an embodiment, the function object 1740 may be connected to an application (e.g., a music player) which may directly play the music. The electronic device 101 may execute an application (or execute an application in the background) in response to a selection (e.g., a touch) of the function object 1740 by the user, and may provide the recommended music to the user by playing the music.

For example, an example is described in FIG. 17B, wherein guidance of the guidance region 1710B relates to a conversation with another user, and a function object 1751, 1753, 1755, or 1760 related to making a call is provided in response thereto. For example, an example of providing the function object 1751, 1753, 1755, or 1760 for inducing making a call to a person to talk to, which is beneficial to the user, according to the guidance of the guidance region 1710B by the electronic device 101 is described. As shown in FIG. 17B, the electronic device 101 may provide, based on the guidance phrase of the guidance region 1710B, a function object 1751, 1753, 1755, or 1760 for inducing making a call to a person to talk to (e.g., another user based on contact information), who made stress and an HR of the user stable, or to be closer to a stable state, while the user was on the phone. According to an embodiment, the function object 1751, 1753, 1755, or 1760 may be connected to an application (e.g., a Phone application) which can directly make a call to another user who has positively affected the user. The electronic device 101 may execute an application (or execute an application in the background) in response to a selection (e.g., a touch) of the function object 1751, 1753, 1755, or 1760, and may attempt to make a call to an electronic device of a user of the selected function object 1751, 1753, 1755, or 1760.

According to an embodiment, the function objects 1751, 1753, 1755, and 1760 may include one or more objects 1751, 1753, and 1755 related to users recommended by the electronic device 101, and a contact object 1760 which allows the user to directly select a person to talk to (or a person to talk on the phone), based on the contact information. According to an embodiment, the function objects 1751, 1753, and 1755 related to the recommended users may be provided based on users each having a high priority among recommended users, wherein as many objects as the number of the recommended users may be provided. The electronic device 101 may provide only the contact object 1760 when there is no recommended user.

FIG. 18 is a flowchart illustrating an operation method of an electronic device according to various embodiments.

Referring to FIG. 18, in operation 1801, the processor 120 of the electronic device 101 may monitor a user context base on context awareness. According to an embodiment, the processor 120 may determine (or check), based on biometric information collected through the context awareness, a stress (or emotional) state of a user. According to an embodiment, the processor 120 may determine a place where the user is located, based on location information collected through the context awareness.

In operation 1803, the processor 120 may detect a context satisfying a condition. According to an embodiment, the processor 120 may determine whether the context of the user is included in a certain condition, based on the result of the monitoring. For example, when it is determined that the stress (or emotional) state of the user has a value higher than a configured condition, the processor 120 may determine that the context satisfies the condition. For example, when the user enters a configured place, the processor 120 may determine that the context satisfies the condition.

In operation 1805, the processor 120 may extract an insight (or an insight card) related to the context. According to an embodiment, the processor 120 may extract an insight appropriate for guiding the user in the user's current context, based on the context awareness. For example, the processor 120 may extract a certain insight related to inducing mind control of the user when the user enters a place in which the stress level of the user was high. For example, when the current stress level of the user increases, the processor 120 may extract a certain insight related to inducing a related action to switch a state of the user to a stable state (e.g., an insight which may induce execution of the function illustrated in FIG. 17A or FIG. 17B).

In operation 1807, the processor 120 may output the extracted insight. According to an embodiment, the processor 120 may determine a scheme which requires the simplest transmission in the user's current context, based on the context awareness, and may output an insight based at least on a tactile, visual, or auditory element, upon the result of the determination.

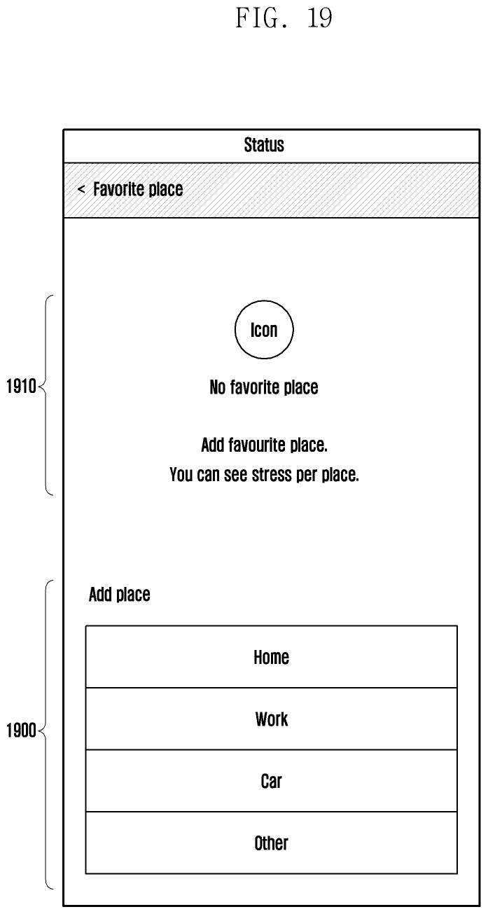

FIGS. 19 to 23 illustrate examples of screens for configuring a user-based place by an electronic device according to various embodiments.

In various embodiments, an interface related to place configuration as shown in FIGS. 19 to 23 may be implemented both in a form of a wearable device or in a form of a smartphone, and may be implemented as an interface corresponding to the form of the electronic device 101. In various embodiments, for convenience of description, an example in which the electronic device 101 provides an interface in a form of a smartphone is described. According to various embodiments, a user may configure (or register) a user's place by using a user account through an interface of the electronic device 101. The place configured by the user may be registered by using the user account through, for example, a server (e.g., the server 603 of FIG. 6).

Referring to FIG. 19, an example of having no place registered by using the user account, or of an initial (or the first) configuration interface for registering a user's place by the user is described. As shown in FIG. 19, in the case of the configuration interface, a basic template 1900 which may be used when the user configures (or adds) a place may be provided. For example, when the template 1900 is provided, place categories (e.g., “Home”, “Work”, or “Car”) which may be registered by the user may be divided into respective taps including an “Other” tap so that the user may register a place other than the given categories, as the user's place. According to various embodiments, based on the template 1900, convenience in registering the user's place can be provided. According to an embodiment, the electronic device 101 may switch to, in response to a selection (e.g., a touch) of each tap of the template 1900, an interface in which a place corresponding to the selected tap may be configured in detail, and may provide the interface to the user.

According to an embodiment, the configuration interface may include a favorite region 1910 for intuitively providing a favorite place (or favorite space) preferred by the user (or the user's favorite place) among places registered by the user. In the example shown in FIG. 19, as there is no place registered by the user, a place related to the favorite region 1910 may not be displayed, and guidance for registering the user's place and adding a favorite place may be provided.

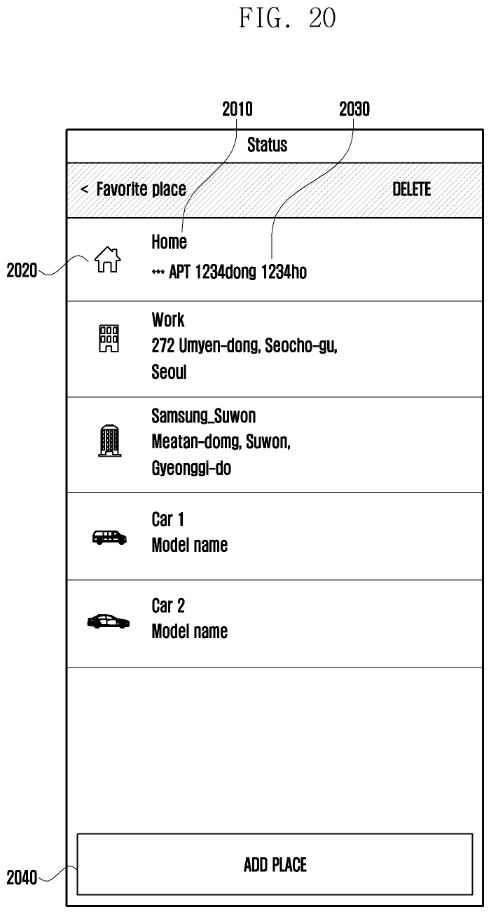

Referring to FIG. 20, an example of an interface for providing information relating to at least one place registered by using a user account is described in FIG. 20. As shown in FIG. 20, an example in the case in which five places are registered by the user is described in FIG. 20. For example, an example showing that the user registers “Home”, “Work”, “Samsung_Suwon”, “Car1”, and “Car2”. As shown in FIG. 20, the interface may configure each item including a place name 2010, a place icon 2020 indicating a category of the place, and a detailed street address 2030 corresponding to the place. According to an embodiment, the interface may include an add tap 2040 for allowing the user to add a place. When the add tap 2040 is selected, the electronic device 101 may switch to an interface including the template 1900 as shown in FIG. 19 and display the switched interface.

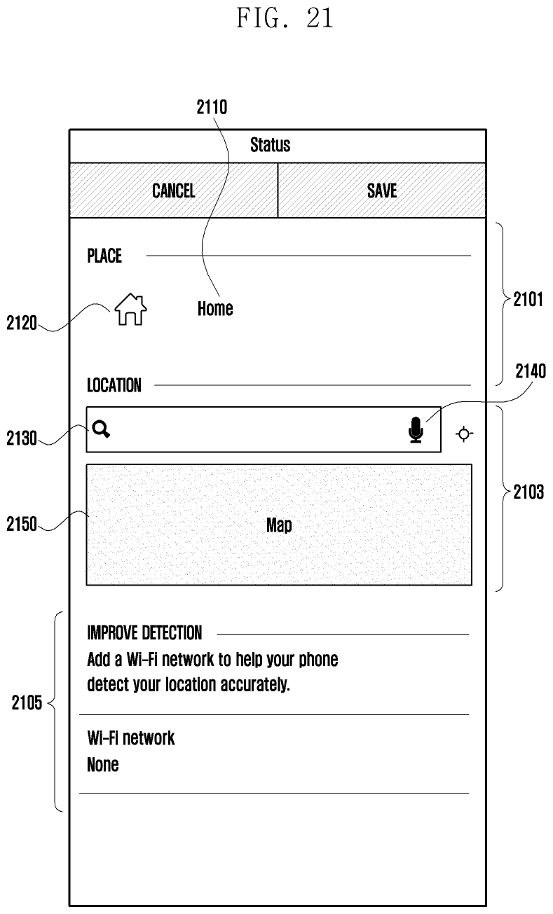

Referring to FIG. 21, an example of a configuration interface in which a specific place may be configured in detail is described in FIG. 21. According to an embodiment, the configuration interface of FIG. 21 may be provided when, for example, the “Home” tap is selected in the template 1900 of FIG. 19, or the “Home” tap is selected in FIG. 20. As shown in FIG. 21, the configuration interface may include a category region 2101 including first information 2110 (e.g., a place name) and second information 2120 (e.g., a place icon) indicating a category of a place selected by the user, a configuration region 2103 related to the detailed location (or street address) configuration on the place, and a network region 2105 for providing guidance related to network connection or configuration (e.g., Wi-Fi connection).

According to an embodiment, the configuration region 2103 may include a search region 2130 (or a search window) in which a detailed location (or street address) of the place may be searched for. In relation to the detailed location search, the search region 2130 may provide a text-input-based search scheme and a voice-input-based search scheme. According to an embodiment, the user may activate a voice input function by selecting (or touching) a microphone object 2140 in the search region 2130 (or while pressing the microphone object 2140), and may search for the detailed location, based on the voice input. According to an embodiment, the configuration region 2103 may include a map region 2150 in which a map for the detailed location found in the search region 2130 may be displayed. According to various embodiments, the user may perform invoking to display the map in the map region 2150, and may search for the detailed location by navigating the displayed map.

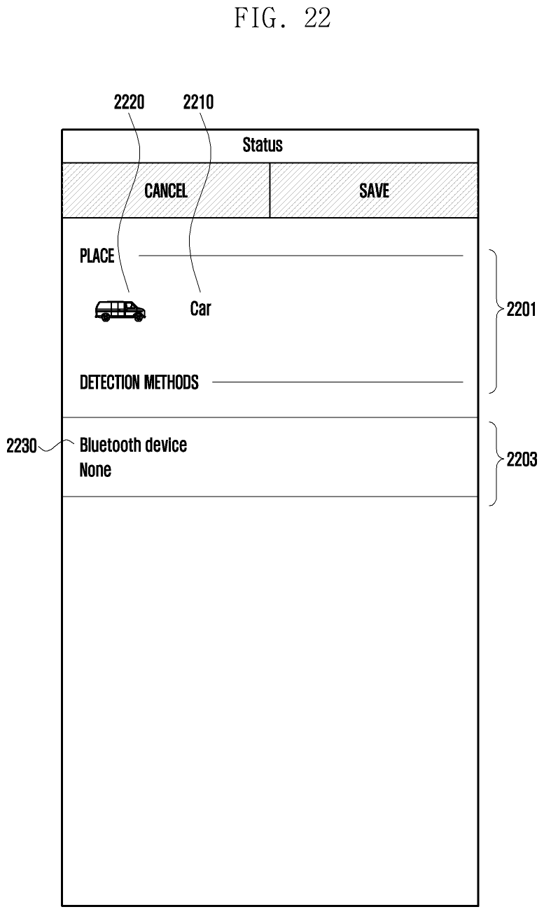

Referring to FIG. 22, another example of a configuration interface in which a specific place may be configured in detail is described in FIG. 22. According to an embodiment, the configuration interface of FIG. 22 may be provided when, for example, the “Car” tap is selected in the template 1900 of FIG. 19, or the “Car” tap is selected in FIG. 20. As shown in FIG. 22, the configuration interface may include a category region 2201 including first information 2210 (e.g., a place name) and second information 2220 (e.g., a place icon) indicating a category of the place selected by the user, and a configuration region 2203 for configuring a scheme of detecting the place.

According to an embodiment, for example, when the category of the place corresponds to “Car”, the configuration region 2203 may indicate a region for configurating a scheme of detecting (or identifying) the corresponding place (e.g., a car of the user). For example, the electronic device 101 may be connected to a communication module provided at the car, according to a configured communication scheme (e.g., Bluetooth communication or direct communication (e.g., wired communication)). Based on car-related identification information acquired at the time of communication with the car, the electronic device 101 may recognize that the user gets in the car, and may detect a place corresponding to the “Car” category.

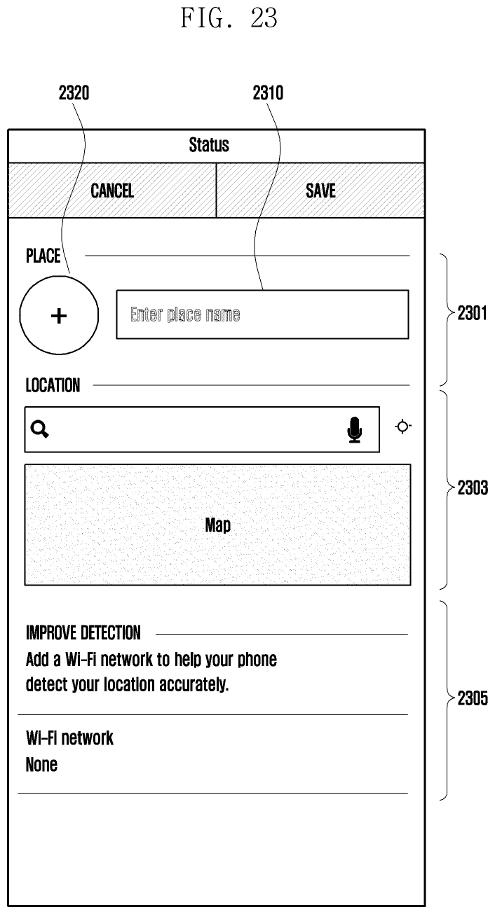

Referring to FIG. 23, another example of a configuration interface in which a specific place may be configured in detail is described in FIG. 23. According to an embodiment, the configuration interface of FIG. 23 may be provided when, for example, the “Other” tap is selected in the template 1900 of FIG. 19. For example, the configuration interface of FIG. 23 may indicate an example of an interface for supporting configuration of a user-defined category (or place) other than the categories according to the template 1900. As shown in FIG. 23, the configuration interface may include a category region 2301 in which a category of a place to be defined by the user may be configured, a configuration region 2303 related to the configuration of the detailed place (or street address) of the place, and a network region 2305 for providing guidance related to network connection or configuration (e.g., Wi-Fi connection).

According to an embodiment, the category region 2301 may include a first region 2310 in which first information (e.g., a place name) may be input, and a second region 2320 in which second information (e.g., a place icon) may be input. According to an embodiment, the second information of the second region 2320 may be configured based on various types of objects (e.g., images, icons, or photos, etc.). According to an embodiment, when the second region 2320 is selected by the user, the electronic device 101 may provide an object interface (e.g., a pop-up window, etc.) in which the user may select an object (e.g., an image, an icon, or a photo, etc.), and may display the object selected from the object interface by the user on the second region 2320, as the second information (e.g., a place icon).

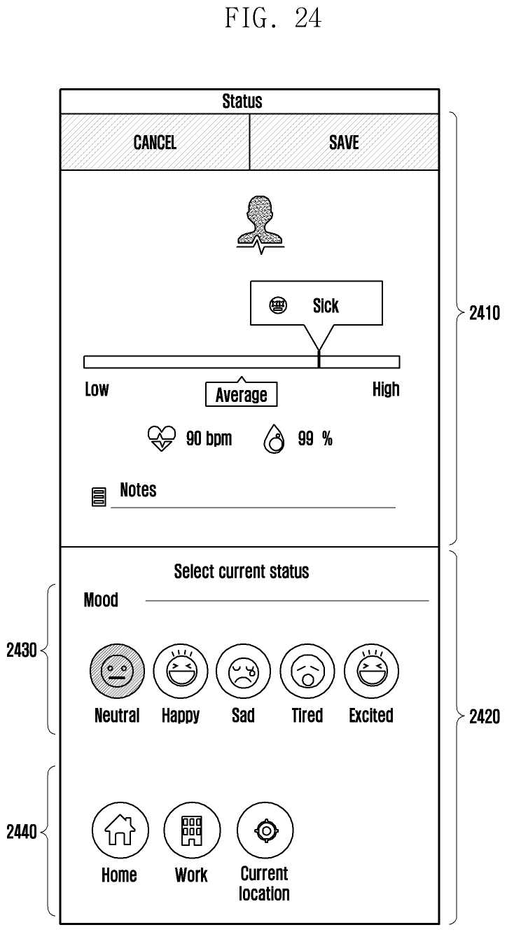

FIG. 24 illustrates an example of a screen for providing biometric information by an electronic device according to various embodiments.

Referring to FIG. 24, an example of an interface including a menu in which the user may directly select (or input) information relating to a user state and information relating to a place after the user measures biometric data by using the electronic device 101 is described in FIG. 24. According to an embodiment, the user may manually input a place, without using automatic location information of the electronic device 101, and may directly input their own state (e.g., emotion or mood) at the time of measuring the biometric data.

As shown in FIG. 24, the interface may include an information region 2410 in which biometric information acquired based on the biometric data is provided, a configuration region 2420 in which, in relation to the biometric information, information relating to the user's current state and information relating to the place may be selected (or input). According to an embodiment, the configuration region 2420 may include a state configuration region 2430 for configuring the user's state and a place configuration region 2440 for configuring a place.

According to an embodiment, the state configuration region 2430 may provide various emotion objects (e.g., emoticons, icons, etc.) related to a state (e.g., emotion or mood) so that the user may directly select the user state. According to an embodiment, the emotion object may be provided in a form of an emoticon and text (e.g., a name) corresponding to, for example, “Neutral”, “Happy”, “Sad, “Tired”, “Excited”, etc. The user may select one emotion object corresponding to the user's current state from among the emotion objects, and the electronic device 101 may match the user state corresponding to the selected emotion object with the biometric information. According to an embodiment, when providing the emotion object, the electronic device 101 may estimate, based on the user's biometric information, the user state, and may provide the emotion object by selecting (or activating or highlighting) the emotion object corresponding to the estimated state. According to an embodiment, the electronic device 101 may provide the emotion object by selecting (or activation or highlighting) the emotion object selected by the user.

According to an embodiment, the place configuration region 2440 may provide a place object (e.g., an icon, etc.) so that the user may directly configure a place where the biometric information is acquired (or a current place). According to an embodiment, the place object may be provided in a form of an icon and text (e.g., a name) for configuring (or designating), for example, home (Home), an office (Work), a current location, etc. The user may select a place object for configuring the user's desired place, or may select a place corresponding to the user's current place, from among the place objects, and the electronic device 101 may match the place corresponding to the selected place object with the biometric information. According to an embodiment, the electronic device 101 may match and store biometric information, the user state, and the place. According to an embodiment, when providing a place object, the electronic device 101 may estimate the user's place based on the user's location information, and may provide the place object by selecting (or activating or highlighting) the place object corresponding to the estimated place. According to an embodiment, when a place (or a location) predesignated by the user is detected, the electronic device 101 may provide the place object by selecting (or activating or highlighting) the corresponding place object. According to an embodiment, the electronic device 101 may provide the place object by selecting (e.g., activation or highlighting) the place object selected by the user.

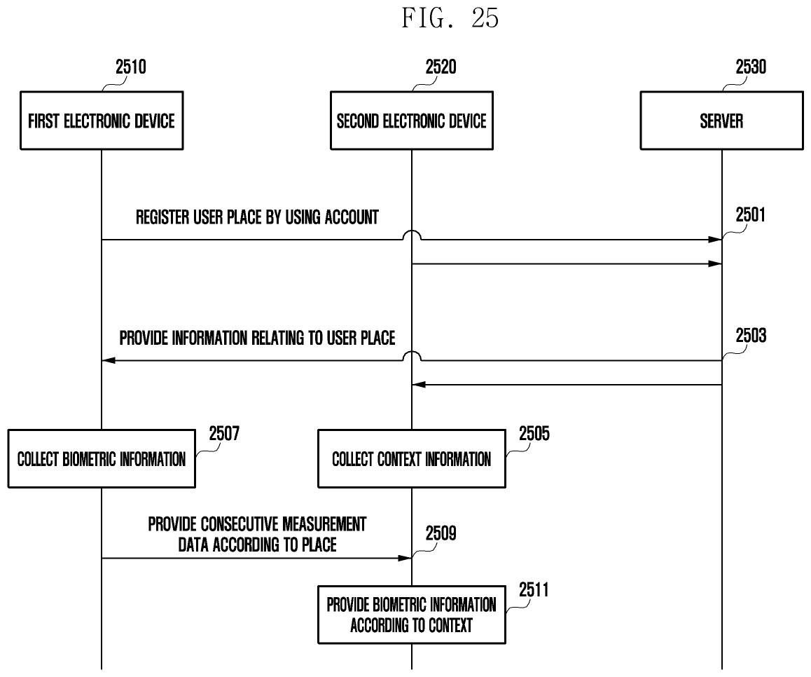

FIG. 25 illustrates an example of providing biometric information based on multiple electronic devices according to various embodiments.

As shown in FIG. 25, an example of configuring biometric information according to various contexts (e.g., places) related to a user, based on interworking between multiple electronic devices (e.g., a first electronic device 2510 and a second electronic device 2520) is described in FIG. 25. For example, an example of measuring biometric data of a user, based on a user's place by the first electronic device 2510, receiving the biometric data from the first electronic device 2510 by the second electronic device 2520, and providing biometric information for each user context or for each user place is described in FIG. 25, wherein the first electronic device 2510 is exemplified by a wearable device, and the second electronic device 2520 is exemplified by a smartphone. According to various embodiments, the first electronic device 2510 and the second electronic device 2520 may include some or all of the electronic device 101 of FIG. 1.

According to various embodiments, a server 2530 may indicate a server for controlling and managing various pieces of information (e.g., personal user information) relating to a user, by using a user account. For example, the server 2530 may include an account server. According to various embodiments, the various pieces of information relating to the user may include information registered to the server 2530 by the user by using the user account (e.g. profile information, device information, health information, place information, or application information, etc.).

Referring to FIG. 25, in operation 2501, the first electronic device 2510 and the second electronic device 2520 may register a user place by using a user account by communicating with the server 2530. According to an embodiment, the user may register the user place to the server 2530 by using at least one of the first electronic device 2510 or the second electronic device 2520. According to various embodiments, a user-defined place configured and registered to the server 2530 by each of the first electronic device 2510 and the second electronic device 2520 may be managed using the user account through the server 2530, and the place managed by using the user account may be synchronized with the first electronic device 2510 and the second electronic device 2520 through the server 2530.

In operation 2503, the server 2530 may provide information relating to the user place to the first electronic device 2510 and the second electronic device 2520. According to an embodiment, the server 2530 may transmit the place managed by using the user account to the first electronic device 2510 and the second electronic device 2520, and the place managed by using the user account may be synchronized with the first electronic device 2510 and the second electronic device 2520 so that the first electronic device 2510 and the second electronic device 2520 have the same information relating to the user place. According to an embodiment, in response to a request for the information relating to the user place from the first electronic device 2510 or the second electronic device 2520, the server 2530 may provide the information relating to the user place to at least one of the electronic devices 2510 and 2520.

In operation 2505, for example, the first electronic device 2510 may be in a state of constantly collecting biometric data of the user in a state while the first electronic device 2510 is worn on the user's body. According to an embodiment, the first electronic device 2510 may acquire (or sense) biometric information related to the user and store the acquired biometric data. According to an embodiment, the first electronic device 2510 may provide related biometric information to the user based at least on the measured biometric data.

In operation 2507, the second electronic device 2520 may be in a state of collecting various pieces of context information.

In operation 2509, the first electronic device 2510 may transmit (or share) the biometric data to (or with) the second electronic device 2520. According to an embodiment, when providing the biometric data, the first electronic device 2510 may also provide information relating to a place where the biometric data is measured. For example, the first electronic device 2510 may transmit the place information and consecutively measured biometric data (or consecutive measurement data) to the second electronic device 2520. According to an embodiment, the first electronic device 2510 may acquire the consecutive measurement data, and may transmit, to the second electronic device 2520, the consecutive measurement data and the place information (or including time information as well) whenever acquiring the consecutive measurement data. According to an embodiment, when the consecutive measurement data is acquired at a configured place, the first electronic device 2510 may transmit, to the second electronic device 2520, the consecutive measurement data and the place information (or including time information as well).

In operation 2511, the second electronic device 2520 may provide biometric information according to a context. According to an embodiment, when providing the biometric information, the second electronic device 2520 may classify the biometric information according to a duration for each place and provide the same. According to an embodiment, the second electronic device 2520 may classify the biometric information according a measured time and/or place, thereby allowing the user to recognize when/where the corresponding result is measured.

According to an embodiment, the second electronic device 2520 may provide the biometric information based at least on biometric data received from the first electronic device 2510, biometric information measured by the second electronic device 2520, or various contexts (e.g., usage logs) related to the use of second electronic device 2520. According to an embodiment, the second electronic device 2520 may recognize (e.g., perform context awareness) and record various usage logs related to the use of the second electronic device 2520 by the user. According to an embodiment, the second electronic device 2520 may monitor and record an application (e.g., an application such as Call, Calendar, Music, Video, or Internet) used by the user by using the second electronic device 2520, or contents (e.g., a call log, a schedule, a music playlist (or item), a video playlist (or item), a web browsing history, etc.) used through the application. According to an embodiment, when monitoring the usage logs, the second electronic device 2520 may store biometric data (or biometric information by biometric data) together with (or in association with or by mapping with) the corresponding usage log.

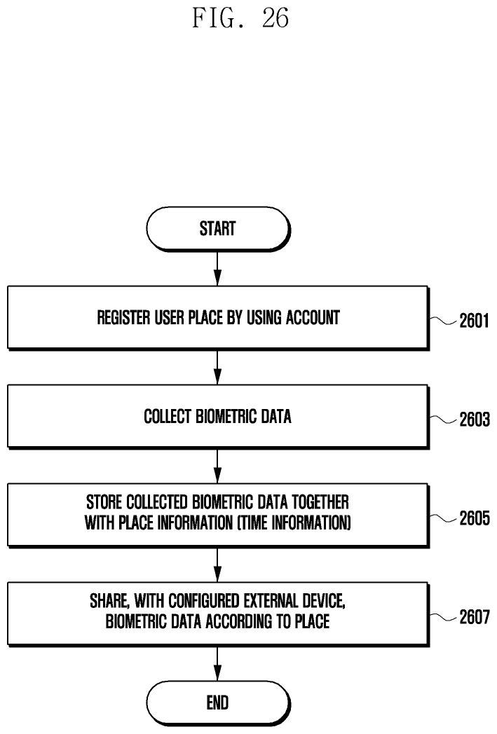

FIG. 26 is a flowchart illustrating an operation method of an electronic device according to various embodiments.

According to various embodiments, an example of the operation of the first electronic device 2510 of FIG. 25 is described in FIG. 26.

Referring to FIG. 26, in operation 2601, a processor (e.g., the processor 120 of the electronic device 101 of FIG. 1) of the electronic device 2510 may register a user place by using an account. According to an embodiment, the processor 120 may communicate with a server (e.g., the server 2530 of FIG. 25) by using a communication module (e.g., the communication module 190 of FIG. 1), and may register a user-defined place to the server 2530 by accessing the server 2530 by using a user account. According to an embodiment, the processor 120 may provide, to the user, the interface described above with reference to FIGS. 19 to 23, and may configure and register a place in response to a user input through the interface.

In operation 2603, the processor 120 may collect biometric data. According to an embodiment, when collecting the biometric data, the processor 120 may include an operation of determining information relating to a place and a time at the corresponding time point.

In operation 2605, the processor 120 may store the collected biometric data together with place information (or including time information as well).

In operation 2607, the processor 120 may share, with a configured external device, biometric information for each place. According to an embodiment, the configured external device may include the electronic device 2510 and another electronic device of the user (e.g., the second electronic device 2520) registered by using the user account.

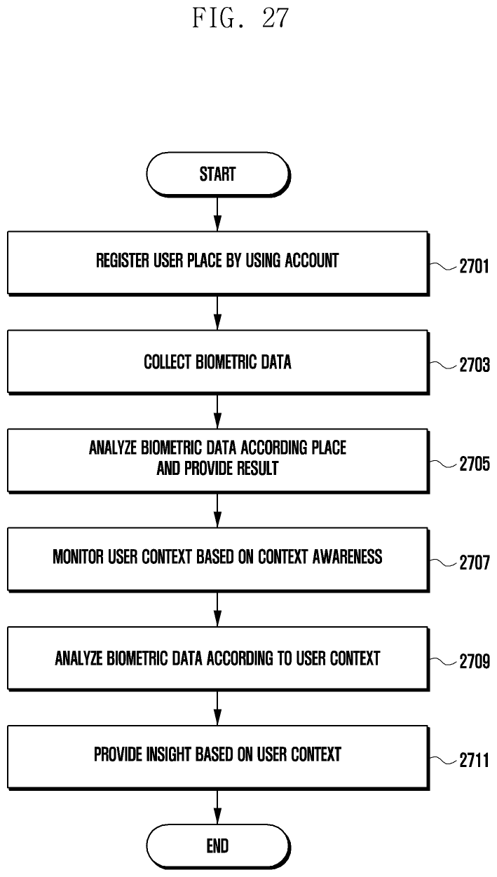

FIG. 27 is a flowchart illustrating an operation method of an electronic device according to various embodiments.

According to various embodiments, an example of the operation of the second electronic device 2520 of FIG. 25 is described in FIG. 27.

Referring to FIG. 27, in operation 2701, a processor (e.g., the processor 120 of the electronic device 101 of FIG. 1) of the electronic device 2520 may register a user place by using an account. According to an embodiment, the processor 120 may communicate with a server (e.g., the server 2530 of FIG. 25) by using a communication module (e.g., the communication module 190 of FIG. 1), and may register a user-defined place to the server 2530 by accessing the server 2530 by using a user account. According to an embodiment, the processor 120 may provide, to the user, the interface described above with reference to FIGS. 19 to 23, and may configure and register a place in response to a user input through the interface.

In operation 2703, the processor 120 may collect biometric information. According to an embodiment, the biometric data may include at least one of biometric data received from an external device (e.g., the first electronic device 2510 of FIG. 25) registered by using the user account, or biometric data collected by the electronic device 2510. According to an embodiment, when first biometric data from the external device is collected together with second biometric data acquired by the electronic device 2510, the processor 120 may synchronize the first biometric data with the second biometric data. According to an embodiment, when the first biometric data and the second biometric data are the same type of biometric data, the processor 120 may match synchronizations of data (e.g., typing, waveform, etc.) between the first biometric data and the second biometric data, and may configure the biometric data as (or may combine into) a single piece of consecutive biometric data. For example, the processor 120 may compare pieces of biometric data by using a feature point (e.g., a peak, a valley, or an inflection point, etc.) of a waveform to verify similarity, and may consider that the pieces of biometric data are the same type of biometric data when the similarity is verified, so as to configure the biometric data as a single piece of consecutive biometric data by combining (or integrating) the pieces of biometric data. According to an embodiment, the processor 120 may acquire place information transmitted together with the biometric data from the external device.

In operation 2705, the processor 120 may analyze biometric data for each place and provide the result of the analysis. According to an embodiment, when the processor 120 may detect a request to display biometric information by a user, or may generate at least one piece of biometric information based on the biometric data, the processor 120 may display the biometric information through a display device (e.g., the display device 160 of FIG. 1) and provide the same to the user. According to various embodiments, when providing the biometric information, the processor 120 may classify the biometric information according to a duration for each place and provide the same. According to an embodiment, the processor 120 may provide the biometric information by classifying the biometric information according to a measured time and/or place, (or according to information on when/where the biometric information is measured), thereby allowing the user to recognize when/where the corresponding result is measured.

In operation 2707, the processor 120 may monitor a user context based on context awareness.

In operation 2709, the processor 120 may analyze biometric data according to the user context. According to an embodiment, the processor 120 may collect biometric data of the user, may determine an amount of changes in the collected biometric data, and the like, and may estimate a user state according to the user context.

In operation 2711, the processor 120 may provide an insight based on the user context. According to an embodiment, with respect to a context in which the user gets negative stress, the processor 120 may provide an insight appropriate for the corresponding context. According to an embodiment, the processor 120 may recommend, to the user, an object positively affecting the user. According to an embodiment, the processor 120 may provide an insight for inducing the user to attempt to make a call to another user (e.g., a family member, a friend, or a person who lowered the stress index of the user when the user talks on the phone with the same person) positively affecting the user. According to an embodiment, the processor 120 may provide an insight (or recommendation or tips) for inducing the user to use an item (e.g., an application, a content, an event, etc.) positively affecting the user.

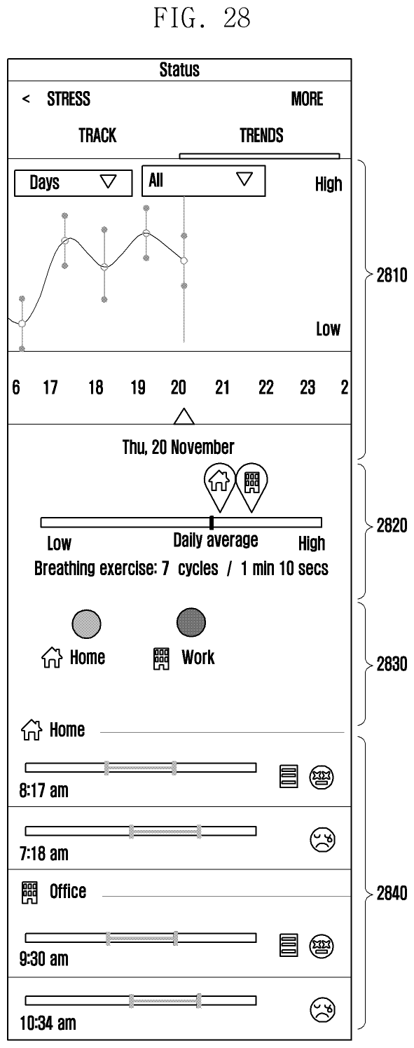

FIGS. 28, 29, and 30 illustrate examples of screens for providing biometric information according a place by an electronic device according to various embodiments.

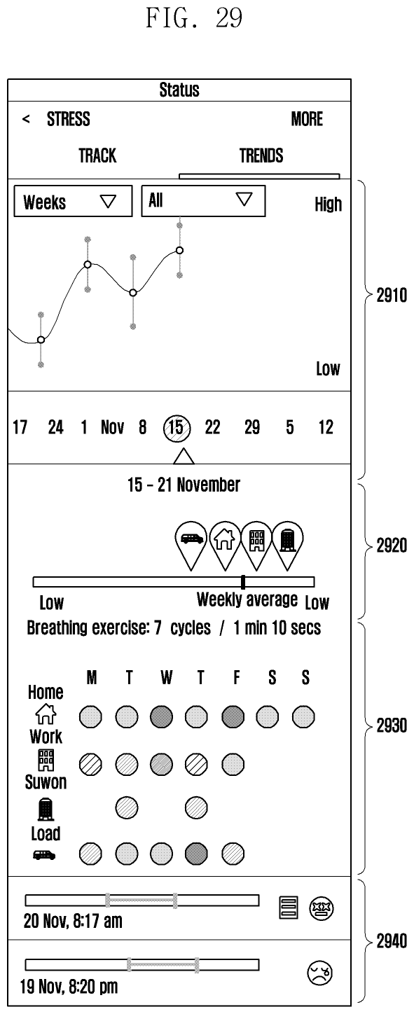

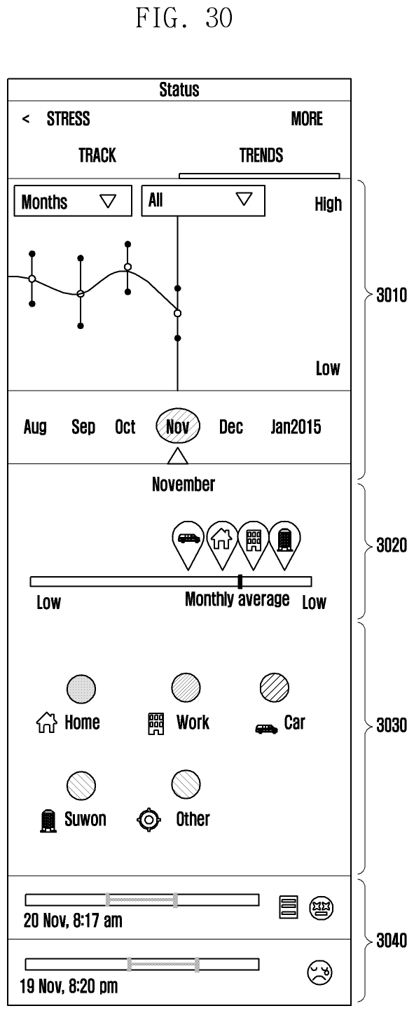

FIGS. 28, 29, and 30 illustrate examples of an interface of providing biometric information by classifying the biometric information according to a place, and may show examples of providing biometric information for each place according to a daily (FIG. 28), weekly (FIG. 29), or monthly (FIG. 30) arrangement reference.

As shown in FIGS. 28, 29, and 30, the interface may include a first region 2810, 2910, or 3010 indicating a chart of biometric information, a second region 2820, 2920, or 3020 indicating a place in which biometric information is measured, a third region 2830, 2930, or 3030 in which place-specific averages of biometric information are represented by colors, and a fourth region 2840, 2940, or 3040 indicating time and state information relating to biometric information for each place.

According to various embodiments, the first region 2810, 2910, or 3010 may include a menu in which a user may select an arrangement reference (e.g. Days, Weeks, or Months), and a chart (e.g., a daily chart, a weekly chart, or a monthly chart) related to the biometric information may be provided according to the selected arrangement reference. According to an embodiment, in the first region 2810, 2910, or 3010, a chart and time information (e.g., date information, week-classification information, or month information) related to the chart may be changed according the arrangement reference and provided. According to an embodiment, the user may select and change a time (e.g., a date, a week unit, or a month) that the user desires to identify for each arrangement reference, from the first region 2810, 2910, or 3010.

According to various embodiments, the second region 2820, 2920, or 3020 may provide place information and an average of pieces of biometric information according to an arrangement reference. According to an embodiment, the second region 2820, 2920, or 3020 may provide information (e.g., a marker) at a position corresponding to the average (e.g., a daily average, a weekly average, or a monthly average) of pieces of biometric information according the arrangement reference in a graph (e.g., a bar graph) indicating the entire duration of the biometric information. According to an embodiment, the second region 2820, 2920, or 3020 may include a place object indicating a place, at a position adjacent to the graph.

According to an embodiment, the place object may be provided at a position, in which the average of pieces of biometric information of each place is recognizable, in the graph. According to an embodiment, the place object may indicate a place where the biometric information is acquired, and may be the number of place objects may vary depending on the daily, weekly, or monthly arrangement reference. For example, the number of places where pieces of biometric information are acquired for a week may be larger than the number of places where pieces of biometric information are acquired for a day. For example, on Monday, a user may move between home and office 1, which are registered places, by using a car that is a registered place, and on Tuesday, a user may move among home, office 1, and office 2, which are registered places, without using a car that is a registered place. The electronic device 101 may acquire biometric information at each place registered by the user, and may store the biometric information by matching the corresponding place with the corresponding biometric information. According to an embodiment, the second region 2820, 2920, or 3020 may further include and provide information relating to the user's breathing exercise.

According to various embodiments, the third region 2830, 2930, or 3030 may provide a color-based average of pieces of biometric information at each place according to an arrangement reference. According to various embodiments, the average of pieces of biometric information may be represented in a predetermined stage of color (e.g., a certain stage among the eight stages shown in FIG. 9), according to a level (or a value) of the biometric information. According to various embodiments, the color corresponding to the average of pieces of biometric information may be the same at each place, or may be different at each place, according to the level of biometric information collected in the corresponding place.

According to an embodiment, FIG. 28 may show an example in which places where pieces of biometric information are collected for a day are home (e.g., Home) and an office (e.g., Work), and color (e.g., green) corresponding to a daily average at home is distinguished from color (e.g., orange) corresponding to a daily average at office.

According to an embodiment, FIG. 29 may show an example in which places where pieces of biometric information are collected for a week are home (e.g., Home), office 1 (e.g., Work), office 2 (e.g., Suwon), and a car (e.g., Load), and day-specific averages of biometric information in a week in each place are distinguished by colors. According to an embodiment, in FIG. 29, when there is no object indicating color (or when there is a blank) at a specific place on a certain day of the week, this may indicate that there is no entrance into the corresponding place by the user, and may indicate that there is no biometric information collected at the corresponding place on the corresponding day.

According to an embodiment, FIG. 30 may show an example in which places where pieces of biometric information are collected for a month are home (e.g., Home), office 1 (e.g., Work), a car (e.g., Car), office 2 (e.g., Suwon), and other places (e.g., Other), and monthly averages of biometric information according to places are distinguished by colors.

According to various embodiments, the fourth area 2840, 2940, or 3040 may provide detailed information relating to the biometric information at each place for each acquisition time (e.g., a time point or a date) and user state (e.g., emotion or mood) information corresponding to each biometric information. According to an embodiment, the state information may include an emotion object indicating a user state (e.g., emotion or mood) at the time of acquiring the biometric information. According to an embodiment, the fourth region 2840, 2940, or 3040 may provide the detailed information according to a time reference or a date reference, depending on an arrangement reference. For example, FIG. 28 may show an example in which daily biometric information is classified and provided according to a time, and FIG. 29 and FIG. 30 may show an example in which weekly or monthly biometric information is classified and provided according to a date (or a day of the week).

As described above, an operation method of an electronic device 101 according to various embodiments may include: acquiring, using the sensor module 176, biometric information of a user and place information related to the user; matching the biometric information with the place information; displaying an interface including biometric information for a predetermined period of time through a display device 160; determining a place of a region selected by the user and a duration corresponding to the place in the interface; and specifying the duration and display biometric information by highlighting the biometric information within the duration in the interface.

According to various embodiments, the matching may include: analyzing a usage log of the electronic device 101; and matching the usage log with biometric information related to the usage log.

According to various embodiments, the operation method of the electronic device 101 may further include outputting an insight corresponding to a user state, based on the biometric information.

According to various embodiments, the outputting of the insight may include: determining a user context based on the biometric information and the place information; and outputting an insight related to the user context.

According to various embodiments, the outputting of the insight may include: determining a user context based on the biometric information and the usage log; and outputting an insight related to the user context.

According to various embodiments, the outputting of the insight may include: determining a user context based at least on the biometric information, the place information, or the usage log; and outputting an insight related to the user context when the user context is included in a configured condition.

According to various embodiments, the operation method of the electronic device 101 may further include: estimating a user state based on biometric information in a specific context related to a user; generating context data related to the user context, based on the user state; and storing the context data.

According to various embodiments, the outputting of the insight may include: analyzing biometric information; determining whether a user state according to the biometric information is included in a configured condition; extracting an insight related to the user state when the user state is included in the configured condition; and outputting the insight.

According to various embodiments, the outputting of the insight may include: performing context awareness when biometric information is collected; and outputting a related insight based on context information according to the context awareness and a user state according to the biometric information.

According to various embodiments, the electronic device 101 may classify biometric information according to a place, and display place-specific averages of biometric information for a predetermined period of time by colors, through the interface, wherein the place includes information registered to a server by using a user account.

The various embodiments of the disclosure described and shown in the specification and the drawings have been presented to easily explain the technical contents of the disclosure and help understanding of the disclosure, and are not intended to limit the scope of the disclosure. Therefore, the scope of the disclosure should be construed to include, in addition to the embodiments disclosed herein, all changes and modifications derived on the basis of the technical idea of the disclosure.

1. An electronic device comprising:
a sensor module;
a display device; and
a processor,
wherein the processor is configured to:
acquire, using the sensor module, biometric information of a user and place information related to the user;
match the biometric information with the place information;
display an interface comprising biometric information for a predetermined period of time through the display device;
determine a place of a region selected by the user and a duration corresponding to the place in the interface; and
specify the duration and display biometric information by highlighting the biometric information within the duration in the interface.

2. The electronic device of claim 1, wherein the processor is configured to:
analyze a usage log of the electronic device; and
match the usage log with biometric information related to the usage log.

3. The electronic device of claim 2, wherein the processor is configured to:
determine a user context based on the biometric information and the place information; and
output an insight related to the user context.

4. The electronic device of claim 2, wherein the processor is configured to:
determine a user context based on the biometric information and the usage log; and
output an insight related to the user context.

5. The electronic device of claim 2, wherein the processor is configured to:
determine a user context based at least on the biometric information, the place information, or the usage log; and
output an insight related to the user context when the user context is comprised in a configured condition.

6. The electronic device of claim 2, wherein the processor is configured to:
estimate a user state based on biometric information in a specific context related to a user;
generate context data related to the user context, based on the user state; and
store the context data.

7. The electronic device of claim 2, wherein the processor is configured to:
analyze biometric information;
determine whether a user state according to the biometric information is comprised in a configured condition;
extract an insight related to the user state when the user state is comprised in the configured condition; and
output the insight.

8. The electronic device of claim 7, wherein the processor is configured to:
perform context awareness when biometric information is collected; and
output a related insight based on context information according to the context awareness and a user state according to the biometric information.

9. The electronic device of claim 2, wherein the place information related to the user comprises information registered to a server by using a user account.

10. The electronic device of claim 2, wherein the processor is configured to classify biometric information according to a place, and display place-specific averages of biometric information for a predetermined period of time by colors, through the interface.

11. An operation method of an electronic device, comprising:
acquiring, using a sensor module, biometric information of a user and place information related to the user;
matching the biometric information with the place information;
displaying an interface comprising biometric information for a predetermined period of time through a display device;
determining a place of a region selected by the user and a duration corresponding to the place in the interface; and
specifying the duration and displaying biometric information by highlighting the biometric information within the duration in the interface.

12. The method of claim 11, wherein the matching comprises:
analyzing a usage log of the electronic device;
matching the usage log with biometric information related to the usage log; and
outputting an insight corresponding to a user state, based on the biometric information.

13. The method of claim 12, wherein the outputting of the insight comprises:
determining a user context based at least on the biometric information, the place information, or the usage log; and
outputting an insight related to the user context when the user context is comprised in a configured condition.

14. The method of claim 12, wherein the outputting of the insight comprises:
analyzing biometric information;
determining whether a user state according to the biometric information is comprised in a configured condition;
extracting an insight related to the user state when the user state is comprised in the configured condition; and
outputting the insight,
wherein the outputting of the insight comprises:
performing context awareness when biometric information is collected; and
outputting a related insight based on context information according to the context awareness and a user state according to the biometric information.

15. The method of claim 12, wherein, through the interface, biometric information is classified according to a place, and place-specific averages of biometric information for a predetermined period of time are displayed by colors, and
wherein the place comprises information registered to a server by using a user account.
